SEQUENCE LISTING (20 sequences)

SEQ ID NO: 1 - DNA, 3606 bp, Homo sapiens
  misc_feature: MAR 1_68 sequence
ctagattataccaacctcataaaataagagcatatataaaagcaaatgctcttatcttgcagatccctgaactgaggaggcaagatcagtttggcagttgaagcagctggaatctgcaattcagagaatctaagaaaagacaaccctgaagagagagacccagaaacctagcaggagtttctccaaacattcaaggctgagggataaatgttacatgcacagggtgagcctccagaggcttgtccattagcaactgctacagtttcattatctcagggatcacagattgtgctacctattgcctaccatctgaaaacagttgcttcctatatttcatccagtttaatatttatttaaaccaagaaggttaatctggcaccagctattccgttgtgagtggatgtgaaagtaccaattccattctgttttactattaactatcctttgccttaatatgtatcagtaggtggcttgttgctaggaaatattaaatgaatggcatgtttcataggttgtgtttaaagttgttttttgagttaaatctttctttaataatactttctgatgtcaaaaacacttagaagtcatggtgttgaacatctatatagggttggatctaaaatagcttcttaacctttcctaaccactgtttttgtttgtttgtttttaactaagcatccagtttgggaaattctgaattaggggaatcataaaaggtttcattttagctgggccacataaggaaagtaagatatcaaattgtaaaaatcgttaagaacttctatcccatctgaagtgtgggttaggtgcctcttctctgtgctcccttaacatcctattttatctgtatatatatatattcttccaaatatccatgcatgggaaaaaaaatctgatcataaaaatattttaggctgggagtggtggctcacgcctgtaatcccagcactttgggaggctgaggtgggcggatcatgaggtcaagagatcgagaccatcctgaccaatatggtgaaaccccatctctactaaagatacaaaactattagctggacgtggtggcacgtgcctgtagtcccagctactcgggaggctgaggcaggagaacggcttgaacccaggaggtggaggttgcagtgagctgagatcgcgccactgcactccagcctgggcgacagagcgagactctgtctcaaaaaaaaaatatatatatatatatatatacacatatatatataaaatatatatatatacacacatatatatataaaatatatatatatacacacatatatataaaatatatatatatacacacatatatataaaatatatatatacacacatatatataaaatatatatatacacacatatatataaaatatatatatacacacatatatataaaatatatatatacacacatatatataaaatatatatatacacacatatatataaaatatatatatacacacatatatataaaatatatatatacacacatatatataaaatatatatatacacacatatatataaaatatatatatacacacatatatataaaatatatatatacacacatatataaaatatatatatacacacatatataaaatatatatatacacatatatataaaatatatatatacacatatatataaaatatatatacacacatatatataaaatatatatatacacacatatatataaaatatatatatacacatatatataaaatatatatatacacatatatataaaatatatatatatacacatatatataaaatatatatacacacatatatataaagtatatatatacacacatatatataaaatatatatatacacatatatataaaatatatatatacacatatatataaaatatatatatacacatatatataaaaatatatatatatattttttaaaatattccaattgtctcactttgtggatgagaaaaagaagtagttagaggtcaagtaacttggcctacatcttttctcaagattgtaaactcctagtgagcaataaccacatcttcattttctttgtataaaacaagaaagtttagcatgaaaaaggtactcaattacaaatgtgttggattgaattgaagacccttggaaggggattttgtacctgaggatctctttcttttggccatattgttcaatggacaaaatttagccttcgaaggcaggccgatttgaggttaatactacctttaccacttgatagctatgtgaccttggccatgtggtttcaacagtctgaacctcattttctctgtgtatgtgtggtcctccttacaagtttgtgaaaaatgtgaagtccttagccatgatagcccaatataacaggctaaatgataataggtttatgttcttttcctttatattctcagataagcactgtccaagtttgaggtgttttgaggtctcgcctgatttggattgtttgagtttatgctattctttgaattctttgagctgttctgaagcagtgtatcatgaacaaaaacatccccagttcagtccaaacccctggttacatatcattcttatgccatgttataaccagtttgagagtgttccctctgttattgcatttaagtttcagcctcacacagaaattcagcagccaatttctaagccctaagcataaaatctggggtgggggggggggatggcctgaagagcagcattatgaatagcaccattataattaatgatctctcaggaagatttacaatcacaggtagcagataaaacaaatagtactgcttctgcacttcccctccttttattcgctatgaaattttatgggaaatcagtccagtgaaaaatgtaagctcttaatctttcccagaaatcctacctcatttgatgaatactttgagggaatgaattagagcatttttttcttttatagtctacttcgcatttacgaagtgaggacggtagcttaggctgcctggccaactgatgagaaggtcagaggcatttttagagacctctgttgtctttcattcatgttcattttccacaaggcaagtaatttccaacaaatcagtgtcttcattagtaataagattattaacaacaataatagtcatagtaactattcagtgagagtccattatatatcaggcattctacaaggtactttatatacatctgagtaaacctcacacaattctacagggaggtatttctatccccatttaacaaataaggaaacgaagtccaagtaaattaacttgcccaaggtcacacagatagtacctggcagaacaggaatttaaacctaaatttgtccaactccaaaagcagccttctatttgttataaatgctgcctctcattatcacatattttattattaacaacaacaaacataccaattagcttaagatacaatacaaccagataatcatgatgacaacagtaattgttatactattataataaaatagatgttttgtatgttactataatcttgaatttgaatagaaatttgcatttctgaaagcatgttcctgtcatctaatatgattctgtatctattaaaatagtactacat

SEQ ID NO: 2 - DNA, 3638 bp, Homo sapiens
  misc_feature: MAR 1_68 construct
gtacccccaaaagaaagagatcctcaggtacaaaatccccttccaagggtcttcaattcaatccaacacatttgtaattgagtacctttttcatgctaaactttcttgttttatacaaagaaaatgaagatgtggttattgctcactaggagtttacaatcttgagaaaagatgtaggccaagttacttgacctctaactacttctttttctcatccacaaagtgagacaattggaatattttaaaaaatatatatatatatttttatatatatgtgtatatatatattttatatatatgtgtatatatatattttatatatatgtgtatatatatattttatatatatgtgtgtatatatatactttatatatatgtgtgtatatatattttatatatatgtgtatatatatatattttatatatatgtgtatatatatattttatatatatgtgtatatatatattttatatatatgtgtgtatatatatattttatatatatgtgtgtatatatattttatatatatgtgtatatatatattttatatatatgtgtatatatatattttatatatgtgtgtatatatatattttatatatgtgtgtatatatatattttatatatatgtgtgtatatatatattttatatatatgtgtgtatatatatattttatatatatgtgtgtatatatatattttatatatatgtgtgtatatatatattttatatatatgtgtgtatatatatattttatatatatgtgtgtatatatatattttatatatatgtgtgtatatatatattttatatatatgtgtgtatatatatattttatatatatgtgtgtatatatatattttatatatatgtgtgtatatatatatattttatatatatgtgtgtatatatatatattttatatatatatgtgtgtatatatatatattttatatatatatgtgtatatatatatatatatatattttttttttgagacagagtctcgctctgtcgcccaggctggagtgcagtggcgcgatctcagctcactgcaacctccacctcctgggttcaagccgttctcctgcctcagcctcccgagtagctgggactacaggcacgtgccaccacgtccagctaatagttttgtatctttagtagagatggggtttcaccatattggtcaggatggtctcgatctcttgacctcatgatccgcccacctcagcctcccaaagtgctgggattacaggcgtgagccaccactcccagcctaaaatatttttatgatcagattttttttcccatgcatggatatttggaagaatatatatatatacagataaaataggatgttaagggagcacagagaagaggcacctaacccacacttcagatgggatagaagttcttaacgatttttacaatttgatatcttactttccttatgtggcccagctaaaatgaaaccttttatgattcccctaattcagaatttcccaaactggatgcttagttaaaaacaaacaaacaaaaacagtggttaggaaaggttaagaagctattttagatccaaccctatatagatgttcaacaccatgacttctaagtgtttttgacatcagaaagtattattaaagaaagatttaactcaaaaaacaactttaaacacaacctatgaaacatgccattcatttaatatttcctagcaacaagccacctactgatacatattaaggcaaaggatagttaatagtaaaacagaatggaattggtactttcacatccactcacaacggaatagctggtgccagattaaccttcttggtttaaataaatattaaactggatgaaatataggaagcaactgttttcagatggtaggcaataggtagcacaatctgtgatccctgagataatgaaactgtagcagttgctaatggacaagcctctggaggctcaccctgtgcatgtaacatttatccctcagccttgaatgtttggagaaactcctgctaggtttctgggtctctctcttcagggttgtcttttcttagattctctgaattgcagattccagctgcttcaactgccaaactgatcttgcctcctcagttcagggatctgcaagataagagcatttgcttttatatatgctcttattttatgaggttggtataatctagctagagtcgagatctttggccatattgttcaatggacaaaatttagccttcgaaggcaggccgatttgaggttaatactacctttaccacttgatagctatgtgaccttggccatgtggtttcaacagtctgaacctcattttctctgtgtatgtgtggtcctccttacaagtttgtgaaaaatgtgaagtccttagccatgatagcccaatataacaggctaaatgataataggtttatgttcttttcctttatattctcagataagcactgtccaagtttgaggtgttttgaggtctcgcctgatttggattgtttgagtttatgctattctttgaattctttgagctgttctgaagcagtgtatcatgaacaaaaacatccccagttcagtccaaacccctggttacatatcattcttatgccatgttataaccagtttgagagtgttccctctgttattgcatttaagtttcagcctcacacagaaattcagcagccaatttctaagccctaagcataaaatctggggtgggggggggggatggcctgaagagcagcattatgaatagcaccattataattaatgatctctcaggaagatttacaatcacaggtagcagataaaacaaatagtactgcttctgcacttcccctccttttattcgctatgaaattttatgggaaatcagtccagtgaaaaatgtaagctcttaatctttcccagaaatcctacctcatttgatgaatactttgagggaatgaattagagcatttttttcttttatagtctacttcgcatttacgaagtgaggacggtagcttaggctgcctggccaactgatgagaaggtcagaggcatttttagagacctctgttgtctttcattcatgttcattttccacaaggcaagtaatttccaacaaatcagtgtcttcattagtaataagattattaacaacaataatagtcatagtaactattcagtgagagtccattatatatcaggcattctacaaggtactttatatacatctgagtaaacctcacacaattctacagggaggtatttctatccccatttaacaaataaggaaacgaagtccaagtaaattaacttgcccaaggtcacacagatagtacctggcagaacaggaatttaaacctaaatttgtccaactccaaaagcagccttctatttgttataaatgctgcctctcattatcacatattttattattaacaacaacaaacataccaattagcttaagatacaatacaaccagataatcatgatgacaacagtaattgttatactattataataaaatagatgttttgtatgttactataatcttgaatttgaatagaaatttgcatttctgaaagcatgttcctgtcatctaatatgattctgtatctattaaaatagtactacatctagccc

SEQ ID NO: 3 - DNA, 5463 bp, Mus musculus
  misc_feature: MAR S4 sequence with full AvaI sites
ctcgaggtctcaagataagaatgactgctgtaactcaaatccaccaaagctatttgtgttagaatgctttcctttggtaataacataataccacagagtgagtgaatgtatcaagcaaagtactcactcataatctctccacccaaatgactttgtcttctaaaattaaacccttcccagaggcctctccccttaataccatattgggctcttcacacttcttccaacatcgccttccatcctggcccttccaacctcccttctgtttgtgctaggaacagctcaaggcctcctatctaccacagagttacatggcttgccccttgccaaccccccagtaccacacagtgagtgcaaaatctcaccacattcagaacccagtcactattcaaatcatattttaacctttgcagtactgactacttttgattcatctaaacattactgaactttattctagaaaacatttaagaaatttgtagttaggttcatcctttgagaccttacatttaatttctttctatgtaaacggaaagcattgttcagtcccacgctcattatggcaacccacttccaagtacttcgtttactacgtgggctggaatcatacagttttctgttgtgcttgtgggagcagatccccctaacctctgctgatttttctcaccacttatcatacatttattacatgcatgcactgctgtgtgagtttctaaatacttgggtagcaattctctactattactttaattttcctacttgtctgcaaatacgaaaagtagcttgaaagaacttcagatctttgttgttatctgttgcaaacactccatttttctgttgtagcaaaaaaaaaaaaaaagacatccatagttgtcaatgagaatgcaagatacatacattctgcacctgtgtgctaacataagtggctgccctgtgactcagagattgcttgtccttctcctaagcctatccttttttgttactttggatacttttgttcaatgaatccagaaaaagtgtttttcagattcaccatgtgaccctcatttaaaacctgtaatccccctatggttaagttcctgcttttgtttctgttttctttctttcagtaaaaggaattgaacccagtccttccacttactatctgagcatatggctcttttagattatgatgttggtggtgttcattggtctcaccaaaatgctaaagaagccttcatcttctacttgtgggtagtctttacattcattactgcaagtttagtttatgtggtagtaccagatcctttgcttcttttgacttcatgcctacctaacagcagctctttcctttagttaagcttatgaaatagtgtttctctcatgtttcctctatattctctcttttgccttcctgtttcttcctgttgattccatcccattggagtgaaatcttatgatcttttggcatcaacaaagtgatctgcatccaaataattccacatctcattccatgttgactgtggatctatatatatatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatacgtatatatgcatatacgtatatatgtatatatgtatatatgtatatatgtatatatgtatatatgtatatatgtatatatgtatatatgtatgtatgtatgtatgtatgtatatatgtatatatgtatgtatgtatgtatgtatgtatgtatgtatatatgtatatatatatgtatgtatgtatgtatgtatgtatgtatatgtgtatatgtgtatatgtgtatatgtgtatatgtgtatatatgtatatatgtatatatgtatatatgtatatgtgtatatgtgtatatgtgtatatatgtatatatgtatatatgtatatatgtatatatataacatagtattaaattatatatacatatataagtgaaatgtcacaatcttctagaacttgctctgtatgtccacttaacatggtagagtgagctatgtcagcattttctatttcctgtgaatcattctgtgtgttgccaagaagaaatatgatatattctgaggttatgaaatgatattttggtcatcatgtttctcatcctattttcatattacctaaatacttttgcttttaaaattattattattaataataatataattatttatacaataatatttaaataatatatttatttaatataattattatatttcacataaaagcaatagttccagtgttacaaattgtaggcaactgggctgttctgattatctaagttgggcccaggatatgtgctgaatagttaaagcacatgcccagcatgtatgagggtaaaaggatgggtggatgtagtgacccatttgtaatttaagccttagcaggcagaggtgtgacccatagtgcaaagtacatagtcattataaggtcatctatatcacaatctctggattagattgattgaacctgctcagtgaccaatgtgttagcaatatacaggaggatgataacatcaacgtcagaagacacattgaagggcttacaaatagtgcccatttactttaatacagaaaaattcaatgtaccctctaggcaatttcaacttttagtctcttggtaggatagtctacatttagaatggctaattcataaattagaaagcttcttcaccccctacttttctggttatttctctatgaatgtggtaggcatgagttagtacacatgtttccatgtacatgtgtttctatgtgtctgcatgcatatggtagaatgtactcatattctatgtacagttagaacaatatttatattgtcaaagaaatcaaaaggagtattataagcttcagaaataaggataagtttgaaatattcattgttttattttttacagtattttttcctttgagaattctatgtaaagtactttgaacatatttgccttcaactcctccctcactttcaccctctcttcattcctccctttcctttccactcaaagttgagattcctttatttatttatttatccttcaaatatcactggtactatccacatgatctcaggattgaggtctgctctgacgtgtcatcctgctttcatgcaatggccttataggtggaacaacattatgaactaaccagtaccccggagctcttgactctagctgcatatatatcaaaagatggcctagtcggccatcactggaaagagaggctcattggacttgcaaactttatatgccccagtacaggggaacaccagggccaaaaagggggagtgggtgggcaggggagtgggggtgggtggatatgggggacttttggtatagcattggaaatgtaaatgagttaaatacctaataaaaaatggaaaaaaaaagtttctaatgtgtgtttctagaaacttcctctcttaaagcaacaacatgtccatgagcaatatagaattgaagatcaccatcaaatcctctttattcctcattgtttccatcatgtactaccagacctctttaaagtgtagtacagtgtgttaggaaatgagcagattatcctgggtatgtgctaaattagctactgagtcaaaatacattttttgctgaacattaagtgtttggtcatttctgggcaaaagaaagaaagaaagaaagaaaagaaagaaagaaaggaaggaaggaaggaaggaaggaaggaaggaaggaaagaaggaaggaaagaaaaaatggatgtaaattgttctgacagcatctgtctgagtcaggcagtggaatgaaggaggaatcctagagaatgcacaggaaagcagcccaaggagagtgtgggctgaaaggcatcatgttagaaacatgcactcgatgacagaaccttgagaaaaaggaactcaagcaaaagcacttatttaaaattgtaaaacgcactttattcatagccatgggggatgtcaatattccaagcataagaatgatcagtttccaatcactgtgaacccccaaaacacaaagtgaaaacccactactttatttgatgagatttggggttgctctattaatttataaaatcagagtaagacacgatataaatgaaacgattgtagttctaaagcagcggcacttccctgaacagtgtcattttgacaagtaactgctaacatcttcaggtcacagcgactgaagaaaaagtagggaaagaaggctggctgtgctgtttgacattttcttttcttatctggtgacatgaagagaagctctgggtccccctactcttgttcatatatctgttgcttttatgctgcatcctgaggtttgaagaaatgcatttggcactgagaaaagatgaggagagaatgccttggacatggtcctaacatgctttggtactgagaaaagagagcagaggagatgacatagaataggagagataatttggcctattttggccttcatctgagtgatagattttacttaacaaatagaaacaaagttttacttataaacagaaccaatgacctgtgtcatctctgatatattgagctttgaattcagtgaaattatgaactaaatatatcactccataattttctaagagggctatttgtatagtttcagtgatagtgtgacaaagtgtaatctaaatttctaaaaagtaaaataagtagataaaatagtaggtagaatagtataataatagaataagtataggtatggactagaataaatagacaaaatagtagataaaatgctaatgattttgttgacagggtaatcatgaatatttttattatttagctaaagaaccaatgttcatgtactcaagaagtgtattgaggaacttaggaaattagtctgaacaggtgagagggtgcgccagagaacctgacagcttctggaacaggcggaagcacagaggcactgaggcagcaccctgtgtgggccggggacagccggccaccttccggaccggaggacaggtgcccgcccggctggggaggcgacctaagccacagcagcagcggtcgccatcttggtccgggacccgccgaacttaggaaattagtctgaacaggtgagagggtgcgccagagaacctgacagcttctggaacaggcagaagcacagaggcgctgaggcagcaccctgtgtgggccggggacagccggccaccttccggaccggaggacaggtgcccacccggctggggaggcggcctaagccacagcagcagcggtcgccatcttggtcccggg

SEQ ID NO: 4 - DNA, 3839 bp, Mus musculus
  misc_feature: S4_1-703_2328-5457 construct with full AvaI sites
ctcgaggtctcaagataagaatgactgctgtaactcaaatccaccaaagctatttgtgttagaatgctttcctttggtaataacataataccacagagtgagtgaatgtatcaagcaaagtactcactcataatctctccacccaaatgactttgtcttctaaaattaaacccttcccagaggcctctccccttaataccatattgggctcttcacacttcttccaacatcgccttccatcctggcccttccaacctcccttctgtttgtgctaggaacagctcaaggcctcctatctaccacagagttacatggcttgccccttgccaaccccccagtaccacacagtgagtgcaaaatctcaccacattcagaacccagtcactattcaaatcatattttaacctttgcagtactgactacttttgattcatctaaacattactgaactttattctagaaaacatttaagaaatttgtagttaggttcatcctttgagaccttacatttaatttctttctatgtaaacggaaagcattgttcagtcccacgctcattatggcaacccacttccaagtacttcgtttactacgtgggctggaatcatacagttttctgttgtgcttgtgggagcagatccccctaacctctgctgatttttctcaccacttatcatacatttattacatgcatgcactgctgtgtgagtttctaaatacttgggtagcaattctctactattactttaattttcctacttgtctgcaaatacgaaaagtagcttgaaagaacttcagatctttgttgttatctgttgcaaacactccatttttctgttgtagcaaaaaaaaaaaaaaagacatccatagttgtcaatgagaatgcaagatacatacattctgcacctgtgtgctaacataagtggctgccctgtgactcagagattgcttgtccttctcctaagcctatccttttttgttactttggatacttttgttcaatgaatccagaaaaagtgtttttcagattcaccatgtgaccctcatttaaaacctgtaatccccctatggttaagttcctgcttttgtttctgttttctttctttcagtaaaaggaattgaacccagtccttccacttactatctgagcatatggctcttttagattatgatgttggtggtgttcattggtctcaccaaaatgctaaagaagccttcatcttctacttgtgggtagtctttacattcattactgcaagtttagtttatgtggtagtaccagatcctttgcttcttttgacttcatgcctacctaacagcagctctttcctttagttaagcttatgaaatagtgtttctctcatgtttcctctatattctctcttttgccttcctgtttcttcctgttgattccatcccattggagtgaaatcttatgatcttttggcatcaacaaagtgatctgcatccaaataattccacatctcattccatgttgactgtggatctatatatatatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatacgtatatatgcatatacgtatatatgtatatatgtatatatgtatatatgtatatatgtatatatgtatatatgtatatatgtatatatgtatgtatgtatgtatgtatgtatatatgtatatatgtatgtatgtatgtatgtatgtatgtatgtatatatgtatatatatatgtatgtatgtatgtatgtatgtatgtatatgtgtatatgtgtatatgtgtatatgtgtatatgtgtatatatgtatatatgtatatatgtatatatgtatatgtgtatatgtgtatatgtgtatatatgtatatatgtatatatgtatatatgtatatatataacatagtattaaattatatatacatatataagtgaaatgtcacaatcttctagaacttgctctgtatgtccacttaacatggtagagtgagctatgtcagcattttctatttcctgtgaatcattctgtgtgttgccaagaagaaatatgatatattctgaggttatgaaatgatattttggtcatcatgtttctcatcctattttcatattacctaaatacttttgcttttaaaattattattattaataataatataattatttatacaataatatttaaataatatatttatttaatataattattatatttcacataaaagcaatagttccagtgttacaaattgtaggcaactgggctgttctgattatctaagttgggcccaggatatgtgctgaatagttaaagcacatgcccagcatgtatgagggtaaaaggatgggtggatgtagtgacccatttgtaatttaagccttagcaggcagaggtgtgacccatagtgcaaagtacatagtcattataaggtcatctatatcacaatctctggattagattgattgaacctgctcagtgaccaatgtgttagcaatatacaggaggatgataacatcaacgtcagaagacacattgaagggcttacaaatagtgcccatttactttaatacagaaaaattcaatgtaccctctaggcaatttcaacttttagtctcttggtaggatagtctacatttagaatggctaattcataaattagaaagcttcttcaccccctacttttctggttatttctctatgaatgtggtaggcatgagttagtacacatgtttccatgtacatgtgtttctatgtgtctgcatgcatatggtagaatgtactcatattctatgtacagttagaacaatatttatattgtcaaagaaatcaaaaggagtattataagcttcagaaataaggataagtttgaaatattcattgttttattttttacagtattttttcctttgagaattcagtgaaattatgaactaaatatatcactccataattttctaagagggctatttgtatagtttcagtgatagtgtgacaaagtgtaatctaaatttctaaaaagtaaaataagtagataaaatagtaggtagaatagtataataatagaataagtataggtatggactagaataaatagacaaaatagtagataaaatgctaatgattttgttgacagggtaatcatgaatatttttattatttagctaaagaaccaatgttcatgtactcaagaagtgtattgaggaacttaggaaattagtctgaacaggtgagagggtgcgccagagaacctgacagcttctggaacaggcggaagcacagaggcactgaggcagcaccctgtgtgggccggggacagccggccaccttccggaccggaggacaggtgcccgcccggctggggaggcgacctaagccacagcagcagcggtcgccatcttggtccgggacccgccgaacttaggaaattagtctgaacaggtgagagggtgcgccagagaacctgacagcttctggaacaggcagaagcacagaggcgctgaggcagcaccctgtgtgggccggggacagccggccaccttccggaccggaggacaggtgcccacccggctggggaggcggcctaagccacagcagcagcggtcgccatcttggtcccggg

SEQ ID NO: 5 - DNA, 3738 bp, Mus musculus
  misc_feature: S4_1-2395_4121-5457 construct with full AvaI sites
ctcgaggtctcaagataagaatgactgctgtaactcaaatccaccaaagctatttgtgttagaatgctttcctttggtaataacataataccacagagtgagtgaatgtatcaagcaaagtactcactcataatctctccacccaaatgactttgtcttctaaaattaaacccttcccagaggcctctccccttaataccatattgggctcttcacacttcttccaacatcgccttccatcctggcccttccaacctcccttctgtttgtgctaggaacagctcaaggcctcctatctaccacagagttacatggcttgccccttgccaaccccccagtaccacacagtgagtgcaaaatctcaccacattcagaacccagtcactattcaaatcatattttaacctttgcagtactgactacttttgattcatctaaacattactgaactttattctagaaaacatttaagaaatttgtagttaggttcatcctttgagaccttacatttaatttctttctatgtaaacggaaagcattgttcagtcccacgctcattatggcaacccacttccaagtacttcgtttactacgtgggctggaatcatacagttttctgttgtgcttgtgggagcagatccccctaacctctgctgatttttctcaccacttatcatacatttattacatgcatgcactgctgtgtgagtttctaaatacttgggtagcaattctctactattactttaattttcctacttgtctgcaaatacgaaaagtagcttgaaagaacttcagatctttgttgttatctgttgcaaacactccatttttctgttgtagcaaaaaaaaaaaaaaagacatccatagttgtcaatgagaatgcaagatacatacattctgcacctgtgtgctaacataagtggctgccctgtgactcagagattgcttgtccttctcctaagcctatccttttttgttactttggatacttttgttcaatgaatccagaaaaagtgtttttcagattcaccatgtgaccctcatttaaaacctgtaatccccctatggttaagttcctgcttttgtttctgttttctttctttcagtaaaaggaattgaacccagtccttccacttactatctgagcatatggctcttttagattatgatgttggtggtgttcattggtctcaccaaaatgctaaagaagccttcatcttctacttgtgggtagtctttacattcattactgcaagtttagtttatgtggtagtaccagatcctttgcttcttttgacttcatgcctacctaacagcagctctttcctttagttaagcttcagaaataaggataagtttgaaatattcattgttttattttttacagtattttttcctttgagaattctatgtaaagtactttgaacatatttgccttcaactcctccctcactttcaccctctcttcattcctccctttcctttccactcaaagttgagattcctttatttatttatttatccttcaaatatcactggtactatccacatgatctcaggattgaggtctgctctgacgtgtcatcctgctttcatgcaatggccttataggtggaacaacattatgaactaaccagtaccccggagctcttgactctagctgcatatatatcaaaagatggcctagtcggccatcactggaaagagaggctcattggacttgcaaactttatatgccccagtacaggggaacaccagggccaaaaagggggagtgggtgggcaggggagtgggggtgggtggatatgggggacttttggtatagcattggaaatgtaaatgagttaaatacctaataaaaaatggaaaaaaaaagtttctaatgtgtgtttctagaaacttcctctcttaaagcaacaacatgtccatgagcaatatagaattgaagatcaccatcaaatcctctttattcctcattgtttccatcatgtactaccagacctctttaaagtgtagtacagtgtgttaggaaatgagcagattatcctgggtatgtgctaaattagctactgagtcaaaatacattttttgctgaacattaagtgtttggtcatttctgggcaaaagaaagaaagaaagaaagaaaagaaagaaagaaaggaaggaaggaaggaaggaaggaaggaaggaaggaaagaaggaaggaaagaaaaaatggatgtaaattgttctgacagcatctgtctgagtcaggcagtggaatgaaggaggaatcctagagaatgcacaggaaagcagcccaaggagagtgtgggctgaaaggcatcatgttagaaacatgcactcgatgacagaaccttgagaaaaaggaactcaagcaaaagcacttatttaaaattgtaaaacgcactttattcatagccatgggggatgtcaatattccaagcataagaatgatcagtttccaatcactgtgaacccccaaaacacaaagtgaaaacccactactttatttgatgagatttggggttgctctattaatttataaaatcagagtaagacacgatataaatgaaacgattgtagttctaaagcagcggcacttccctgaacagtgtcattttgacaagtaactgctaacatcttcaggtcacagcgactgaagaaaaagtagggaaagaaggctggctgtgctgtttgacattttcttttcttatctggtgacatgaagagaagctctgggtccccctactcttgttcatatatctgttgcttttatgctgcatcctgaggtttgaagaaatgcatttggcactgagaaaagatgaggagagaatgccttggacatggtcctaacatgctttggtactgagaaaagagagcagaggagatgacatagaataggagagataatttggcctattttggccttcatctgagtgatagattttacttaacaaatagaaacaaagttttacttataaacagaaccaatgacctgtgtcatctctgatatattgagctttgaattcagtgaaattatgaactaaatatatcactccataattttctaagagggctatttgtatagtttcagtgatagtgtgacaaagtgtaatctaaatttctaaaaagtaaaataagtagataaaatagtaggtagaatagtataataatagaataagtataggtatggactagaataaatagacaaaatagtagataaaatgctaatgattttgttgacagggtaatcatgaatatttttattatttagctaaagaaccaatgttcatgtactcaagaagtgtattgaggaacttaggaaattagtctgaacaggtgagagggtgcgccagagaacctgacagcttctggaacaggcggaagcacagaggcactgaggcagcaccctgtgtgggccggggacagccggccaccttccggaccggaggacaggtgcccgcccggctggggaggcgacctaagccacagcagcagcggtcgccatcttggtccgggacccgccgaacttaggaaattagtctgaacaggtgagagggtgcgccagagaacctgacagcttctggaacaggcagaagcacagaggcgctgaggcagcaccctgtgtgggccggggacagccggccaccttccggaccggaggacaggtgcccacccggctggggaggcggcctaagccacagcagcagcggtcgccatcttggtcccggg

SEQ ID NO: 6 - DNA, 3136 bp, Mus musculus
  misc_feature: S4_2328-5457 construct with full AvaI and EcoRI sites
ctcgaggtctcaagataagaatgactgctgtaactcaaatccaccaaagctatttgtgttagaatgctttcctttggtaataacataataccacagagtgagtgaatgtatcaagcaaagtactcactcataatctctccacccaaatgactttgtcttctaaaattaaacccttcccagaggcctctccccttaataccatattgggctcttcacacttcttccaacatcgccttccatcctggcccttccaacctcccttctgtttgtgctaggaacagctcaaggcctcctatctaccacagagttacatggcttgccccttgccaaccccccagtaccacacagtgagtgcaaaatctcaccacattcagaacccagtcactattcaaatcatattttaacctttgcagtactgactacttttgattcatctaaacattactgaactttattctagaaaacatttaagaaatttgtagttaggttcatcctttgagaccttacatttaatttctttctatgtaaacggaaagcattgttcagtcccacgctcattatggcaacccacttccaagtacttcgtttactacgtgggctggaatcatacagttttctgttgtgcttgtgggagcagatccccctaacctctgctgatttttctcaccacttatcatacatttattacatgcatgcactgctgtgtgagtttctaaatacttgggtagcaattctctactattactttaattttcctacttgtctgcaaatacgaaaagtagcttgaaagaacttcagatctttgttgttatctgttgcaaacactccatttttctgttgtagcaaaaaaaaaaaaaaagacatccatagttgtcaatgagaatgcaagatacatacattctgcacctgtgtgctaacataagtggctgccctgtgactcagagattgcttgtccttctcctaagcctatccttttttgttactttggatacttttgttcaatgaatccagaaaaagtgtttttcagattcaccatgtgaccctcatttaaaacctgtaatccccctatggttaagttcctgcttttgtttctgttttctttctttcagtaaaaggaattgaacccagtccttccacttactatctgagcatatggctcttttagattatgatgttggtggtgttcattggtctcaccaaaatgctaaagaagccttcatcttctacttgtgggtagtctttacattcattactgcaagtttagtttatgtggtagtaccagatcctttgcttcttttgacttcatgcctacctaacagcagctctttcctttagttaagcttatgaaatagtgtttctctcatgtttcctctatattctctcttttgccttcctgtttcttcctgttgattccatcccattggagtgaaatcttatgatcttttggcatcaacaaagtgatctgcatccaaataattccacatctcattccatgttgactgtggatctatatatatatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatacgtatatatgcatatacgtatatatgtatatatgtatatatgtatatatgtatatatgtatatatgtatatatgtatatatgtatatatgtatgtatgtatgtatgtatgtatatatgtatatatgtatgtatgtatgtatgtatgtatgtatgtatatatgtatatatatatgtatgtatgtatgtatgtatgtatgtatatgtgtatatgtgtatatgtgtatatgtgtatatgtgtatatatgtatatatgtatatatgtatatatgtatatgtgtatatgtgtatatgtgtatatatgtatatatgtatatatgtatatatgtatatatataacatagtattaaattatatatacatatataagtgaaatgtcacaatcttctagaacttgctctgtatgtccacttaacatggtagagtgagctatgtcagcattttctatttcctgtgaatcattctgtgtgttgccaagaagaaatatgatatattctgaggttatgaaatgatattttggtcatcatgtttctcatcctattttcatattacctaaatacttttgcttttaaaattattattattaataataatataattatttatacaataatatttaaataatatatttatttaatataattattatatttcacataaaagcaatagttccagtgttacaaattgtaggcaactgggctgttctgattatctaagttgggcccaggatatgtgctgaatagttaaagcacatgcccagcatgtatgagggtaaaaggatgggtggatgtagtgacccatttgtaatttaagccttagcaggcagaggtgtgacccatagtgcaaagtacatagtcattataaggtcatctatatcacaatctctggattagattgattgaacctgctcagtgaccaatgtgttagcaatatacaggaggatgataacatcaacgtcagaagacacattgaagggcttacaaatagtgcccatttactttaatacagaaaaattcaatgtaccctctaggcaatttcaacttttagtctcttggtaggatagtctacatttagaatggctaattcataaattagaaagcttcttcaccccctacttttctggttatttctctatgaatgtggtaggcatgagttagtacacatgtttccatgtacatgtgtttctatgtgtctgcatgcatatggtagaatgtactcatattctatgtacagttagaacaatatttatattgtcaaagaaatcaaaaggagtattataagcttcagaaataaggataagtttgaaatattcattgttttattttttacagtattttttcctttgagaattc

SEQ ID NO: 7 - DNA, 2340 bp, Mus musculus
  misc_feature: S4_2328-4661 construct with full AvaI and BglII sites
agatctttgttgttatctgttgcaaacactccatttttctgttgtagcaaaaaaaaaaaaaaagacatccatagttgtcaatgagaatgcaagatacatacattctgcacctgtgtgctaacataagtggctgccctgtgactcagagattgcttgtccttctcctaagcctatccttttttgttactttggatacttttgttcaatgaatccagaaaaagtgtttttcagattcaccatgtgaccctcatttaaaacctgtaatccccctatggttaagttcctgcttttgtttctgttttctttctttcagtaaaaggaattgaacccagtccttccacttactatctgagcatatggctcttttagattatgatgttggtggtgttcattggtctcaccaaaatgctaaagaagccttcatcttctacttgtgggtagtctttacattcattactgcaagtttagtttatgtggtagtaccagatcctttgcttcttttgacttcatgcctacctaacagcagctctttcctttagttaagcttatgaaatagtgtttctctcatgtttcctctatattctctcttttgccttcctgtttcttcctgttgattccatcccattggagtgaaatcttatgatcttttggcatcaacaaagtgatctgcatccaaataattccacatctcattccatgttgactgtggatctatatatatatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatacgtatatatgcatatacgtatatatgtatatatgtatatatgtatatatgtatatatgtatatatgtatatatgtatatatgtatatatgtatgtatgtatgtatgtatgtatatatgtatatatgtatgtatgtatgtatgtatgtatgtatgtatatatgtatatatatatgtatgtatgtatgtatgtatgtatgtatatgtgtatatgtgtatatgtgtatatgtgtatatgtgtatatatgtatatatgtatatatgtatatatgtatatgtgtatatgtgtatatgtgtatatatgtatatatgtatatatgtatatatgtatatatataacatagtattaaattatatatacatatataagtgaaatgtcacaatcttctagaacttgctctgtatgtccacttaacatggtagagtgagctatgtcagcattttctatttcctgtgaatcattctgtgtgttgccaagaagaaatatgatatattctgaggttatgaaatgatattttggtcatcatgtttctcatcctattttcatattacctaaatacttttgcttttaaaattattattattaataataatataattatttatacaataatatttaaataatatatttatttaatataattattatatttcacataaaagcaatagttccagtgttacaaattgtaggcaactgggctgttctgattatctaagttgggcccaggatatgtgctgaatagttaaagcacatgcccagcatgtatgagggtaaaaggatgggtggatgtagtgacccatttgtaatttaagccttagcaggcagaggtgtgacccatagtgcaaagtacatagtcattataaggtcatctatatcacaatctctggattagattgattgaacctgctcagtgaccaatgtgttagcaatatacaggaggatgataacatcaacgtcagaagacacattgaagggcttacaaatagtgcccatttactttaatacagaaaaattcaatgtaccctctaggcaatttcaacttttagtctcttggtaggatagtctacatttagaatggctaattcataaattagaaagcttcttcaccccctacttttctggttatttctctatgaatgtggtaggcatgagttagtacacatgtttccatgtacatgtgtttctatgtgtctgcatgcatatggtagaatgtactcatattctatgtacagttagaacaatatttatattgtcaaagaaatcaaaaggagtattataagcttcagaaataaggataagtttgaaatattcattgttttattttttacagtattttttcctttgagaattc

SEQ ID NO: 8 - DNA, 4667 bp, Mus musculus
  misc_feature: S4_1-4661 construct with full AvaI and BglII sites
agatctttgttgttatctgttgcaaacactccatttttctgttgtagcaaaaaaaaaaaaaaagacatccatagttgtcaatgagaatgcaagatacatacattctgcacctgtgtgctaacataagtggctgccctgtgactcagagattgcttgtccttctcctaagcctatccttttttgttactttggatacttttgttcaatgaatccagaaaaagtgtttttcagattcaccatgtgaccctcatttaaaacctgtaatccccctatggttaagttcctgcttttgtttctgttttctttctttcagtaaaaggaattgaacccagtccttccacttactatctgagcatatggctcttttagattatgatgttggtggtgttcattggtctcaccaaaatgctaaagaagccttcatcttctacttgtgggtagtctttacattcattactgcaagtttagtttatgtggtagtaccagatcctttgcttcttttgacttcatgcctacctaacagcagctctttcctttagttaagcttatgaaatagtgtttctctcatgtttcctctatattctctcttttgccttcctgtttcttcctgttgattccatcccattggagtgaaatcttatgatcttttggcatcaacaaagtgatctgcatccaaataattccacatctcattccatgttgactgtggatctatatatatatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatatgtatatacgtatatatgcatatacgtatatatgtatatatgtatatatgtatatatgtatatatgtatatatgtatatatgtatatatgtatatatgtatgtatgtatgtatgtatgtatatatgtatatatgtatgtatgtatgtatgtatgtatgtatgtatatatgtatatatatatgtatgtatgtatgtatgtatgtatgtatatgtgtatatgtgtatatgtgtatatgtgtatatgtgtatatatgtatatatgtatatatgtatatatgtatatgtgtatatgtgtatatgtgtatatatgtatatatgtatatatgtatatatgtatatatataacatagtattaaattatatatacatatataagtgaaatgtcacaatcttctagaacttgctctgtatgtccacttaacatggtagagtgagctatgtcagcattttctatttcctgtgaatcattctgtgtgttgccaagaagaaatatgatatattctgaggttatgaaatgatattttggtcatcatgtttctcatcctattttcatattacctaaatacttttgcttttaaaattattattattaataataatataattatttatacaataatatttaaataatatatttatttaatataattattatatttcacataaaagcaatagttccagtgttacaaattgtaggcaactgggctgttctgattatctaagttgggcccaggatatgtgctgaatagttaaagcacatgcccagcatgtatgagggtaaaaggatgggtggatgtagtgacccatttgtaatttaagccttagcaggcagaggtgtgacccatagtgcaaagtacatagtcattataaggtcatctatatcacaatctctggattagattgattgaacctgctcagtgaccaatgtgttagcaatatacaggaggatgataacatcaacgtcagaagacacattgaagggcttacaaatagtgcccatttactttaatacagaaaaattcaatgtaccctctaggcaatttcaacttttagtctcttggtaggatagtctacatttagaatggctaattcataaattagaaagcttcttcaccccctacttttctggttatttctctatgaatgtggtaggcatgagttagtacacatgtttccatgtacatgtgtttctatgtgtctgcatgcatatggtagaatgtactcatattctatgtacagttagaacaatatttatattgtcaaagaaatcaaaaggagtattataagcttcagaaataaggataagtttgaaatattcattgttttattttttacagtattttttcctttgagaattctatgtaaagtactttgaacatatttgccttcaactcctccctcactttcaccctctcttcattcctccctttcctttccactcaaagttgagattcctttatttatttatttatccttcaaatatcactggtactatccacatgatctcaggattgaggtctgctctgacgtgtcatcctgctttcatgcaatggccttataggtggaacaacattatgaactaaccagtaccccggagctcttgactctagctgcatatatatcaaaagatggcctagtcggccatcactggaaagagaggctcattggacttgcaaactttatatgccccagtacaggggaacaccagggccaaaaagggggagtgggtgggcaggggagtgggggtgggtggatatgggggacttttggtatagcattggaaatgtaaatgagttaaatacctaataaaaaatggaaaaaaaaagtttctaatgtgtgtttctagaaacttcctctcttaaagcaacaacatgtccatgagcaatatagaattgaagatcaccatcaaatcctctttattcctcattgtttccatcatgtactaccagacctctttaaagtgtagtacagtgtgttaggaaatgagcagattatcctgggtatgtgctaaattagctactgagtcaaaatacattttttgctgaacattaagtgtttggtcatttctgggcaaaagaaagaaagaaagaaagaaaagaaagaaagaaaggaaggaaggaaggaaggaaggaaggaaggaaggaaagaaggaaggaaagaaaaaatggatgtaaattgttctgacagcatctgtctgagtcaggcagtggaatgaaggaggaatcctagagaatgcacaggaaagcagcccaaggagagtgtgggctgaaaggcatcatgttagaaacatgcactcgatgacagaaccttgagaaaaaggaactcaagcaaaagcacttatttaaaattgtaaaacgcactttattcatagccatgggggatgtcaatattccaagcataagaatgatcagtttccaatcactgtgaacccccaaaacacaaagtgaaaacccactactttatttgatgagatttggggttgctctattaatttataaaatcagagtaagacacgatataaatgaaacgattgtagttctaaagcagcggcacttccctgaacagtgtcattttgacaagtaactgctaacatcttcaggtcacagcgactgaagaaaaagtagggaaagaaggctggctgtgctgtttgacattttcttttcttatctggtgacatgaagagaagctctgggtccccctactcttgttcatatatctgttgcttttatgctgcatcctgaggtttgaagaaatgcatttggcactgagaaaagatgaggagagaatgccttggacatggtcctaacatgctttggtactgagaaaagagagcagaggagatgacatagaataggagagataatttggcctattttggccttcatctgagtgatagattttacttaacaaatagaaacaaagttttacttataaacagaaccaatgacctgtgtcatctctgatatattgagctttgaattcagtgaaattatgaactaaatatatcactccataattttctaagagggctatttgtatagtttcagtgatagtgtgacaaagtgtaatctaaatttctaaaaagtaaaataagtagataaaatagtaggtagaatagtataataatagaataagtataggtatggactagaataaatagacaaaatagtagataaaatgctaatgattttgttgacagggtaatcatgaatatttttattatttagctaaagaaccaatgttcatgtactcaagaagtgtattgaggaacttaggaaattagtctgaacaggtgagagggtgcgccagagaacctgacagcttctggaacaggcggaagcacagaggcactgaggcagcaccctgtgtgggccggggacagccggccaccttccggaccggaggacaggtgcccgcccggctggggaggcgacctaagccacagcagcagcggtcgccatcttggtccgggacccgccgaacttaggaaattagtctgaacaggtgagagggtgcgccagagaacctgacagcttctggaacaggcagaagcacagaggcgctgaggcagcaccctgtgtgggccggggacagccggccaccttccggaccggaggacaggtgcccacccggctggggaggcggcctaagccacagcagcagcggtcgccatcttggtcccggg

SEQ ID NO: 9 - DNA, 802 bp, Mus musculus
  misc_feature: S4_4662-5457 construct with full AvaI and BglII sites
ctcgaggtctcaagataagaatgactgctgtaactcaaatccaccaaagctatttgtgttagaatgctttcctttggtaataacataataccacagagtgagtgaatgtatcaagcaaagtactcactcataatctctccacccaaatgactttgtcttctaaaattaaacccttcccagaggcctctccccttaataccatattgggctcttcacacttcttccaacatcgccttccatcctggcccttccaacctcccttctgtttgtgctaggaacagctcaaggcctcctatctaccacagagttacatggcttgccccttgccaaccccccagtaccacacagtgagtgcaaaatctcaccacattcagaacccagtcactattcaaatcatattttaacctttgcagtactgactacttttgattcatctaaacattactgaactttattctagaaaacatttaagaaatttgtagttaggttcatcctttgagaccttacatttaatttctttctatgtaaacggaaagcattgttcagtcccacgctcattatggcaacccacttccaagtacttcgtttactacgtgggctggaatcatacagttttctgttgtgcttgtgggagcagatccccctaacctctgctgatttttctcaccacttatcatacatttattacatgcatgcactgctgtgtgagtttctaaatacttgggtagcaattctctactattactttaattttcctacttgtctgcaaatacgaaaagtagcttgaaagaacttcagatct

SEQ ID NO: 10 - DNA, 3970 bp, Mus musculus
  misc_feature: MAR S46 sequence with full BamHI sites
ggatccagagcagatgacacatacatatttctcttagatgatattatctgagtgttaagtactaaaatgttgtgtgttgccttatttacattaaacacatttcccttttcactttttttttttcaaactcacttaaaaatgagaggataataaaacggaaactcttcaaagcattttctggtagagatgcagaggaaaaaaaatggtatttcatcaactgatgaaattacttagatctaagtgcatcaccatctaaaactacctacctctttaaagcttcagtatagaaatatttcaaactattttttgaggtatgcttttaaaatgggtttatttactagtatatatacatgcatttaagagtgtttgtggagattagctagaggttgaattgggacactctgttctcaccttctaccacatgagtcccagaggttgcttaggttgagaagttctgcagcaaacacatttacacacggagcaatcccagtagccctcacactttgcaatgagcttgagagttagagcccagcgtgagctgactcatgcctttccattatgtctaaattccaatggcgttttaaaacatttttttatatagcaaaaccacatatgattgggattaaaactgtcaagcagaaatatgaataacttttttcacttaaatttcgtattttatctgaaattttgaccttagaaatacttgacattatatctcaataaaactggcaatgaggaaaaatgaattattggtttagaggttggtcttattattgcttgatacattaacaggagacacttactagggcttatcactgaagtcacccggtacaaatgtacctaagtgaccgagtctagaaaacaggcactcagatactggaggttgaagaagcagcttgcccaatcaatgctctaattccaattttatattcttcctgcctatattagttttccttaagcatagcgagctgaaaaaatgactgtggccttatacatatcctacaggtcaacatgatgaatggctgagttggagttttgaaaaggtgtgaatcacaagactgcgtctggctggatgttgatacctccccaatcccatgactttgtggggacgtggcattcatctctcacagagtaatgtgcagttctcagttcatgggtggctacgaactgaactcccacagtttatcacatacattcttgtgatgtcttgcaatttgttttcgttgtttgttgagtgtgggtatttgagggacaccatgtgtgtagtcagcacatgcatgtgcttctatctggagttggtattcattgtgtgtcctctctctctctttctctctctctctctctctctctctctctctctctctctctctctctctctctctctctctttgtgtgtgtgtgtgtgtgtgtgtgtgtgtgtcttacctgccactggcctcaataatagtagcttagttgggtagtgtgacccagacatcagtctgttttgtctctggtgatggaatcatgctattttgcactgtcaagtgattagtcatttctgagggtcagactcaggtccccatacttccaatataaattgctccccaatggcaaatttctacataacatgaggtcctttctgtagaactgcacaggaaatgacacccattctttctggcaattagtaatgcaagatggaatatgcaaaagcagggaacaagcccagaagtcaatactacttttaaggattttgaaagaaaattgtcattaacgtgccttctcttttataaaagtaagaaaactaaggcccattcttagggacaaggattaattgtccattatcttaagaggagaattataatcatatatgaatttgtgattttattatcacgaagaaactacacacaaatacttctgtttttcattgattccttattgaaccaatattgagttgtgtttctttggactctgtacatacacttacagaagaaatagaatagaagtgacactgaaaatttactgtgcatgtttttcattggaaagcattacaatcatttaagggaacaatgcatttgatagaaacttcagatatcatacacatgttctgatacagaggaattaagtatgcatttcattaaaatagtgttccttgcatataatcattcattaggtcttaaataagatattgttattaacatttaacaaacaataaggttacctaatccagaactgcatgatgataatgacctgaggacacaacaaagtagatggttgaaggttcacaagcccaacccctagatggctagggagagaaggagaatcttgttctccagggatgcggtgcctgataggttgtccagatttagcctgaataaaacatatataataataactctaaatgcattcagtaagttctcaatatgtatatatgtatatatgtatatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatacatatatatatgcacttatatgtgataatagcaattataagaaaagatatctgactttaaaagagattttatgagaggagttggagggataataggaagatggaaatactgaaactatagtgtgaagtatatgtataaatatatatatatgttatacatgtaaatatatatgatatgatatatagatcaagatcatatcagattataatattgtgtcttttaaatttccatgagatgaggatttcaaggctgagtaaactcttttttttaatattttttattataacgtattttcctcaattacatttagaatgctatcccaaaagtcccccataccctccccccaacttccctacccacccattcccattttttggccctggcattcccctgtactgggacatataaagtttgcgtgtccaatgggtctctgtttccagcaatggccgactaggccatcttttgatacatatgcagctagagtcaagagctccggggtactggttagttcataatgttgttgcacctacagggttgcagatctcttaagtccttggatactttctctagctcctccgttgggggcactatgcaccatccaatagctgactgtgagcatctacttatgtgtttgctaggcctggcctagtctcacaagagacagctatatcagggtcctttcagcaaaatcttgctagtgtatgcaatggtttcatcgtttggaggctaattatgggatggatctctggatatggcagtctctagatggtccatccttttgtctcggctccaaactttgctcagcatccttattcatcagagaaatgcaaatcaaaaccctgagataccatctcacaccagtcagaatagctaagatcaaaaattcaggtgacagcagatgttggcgaggatgtggagaaagaggaacactcctccattgttggtgggattgcaagcttgtacaaccactctggaaatcagtctggcggttcctcagaaaattggacatagtactactggaggatcc

SEQ ID NO: 11 - DNA, 30 bp, Artificial
  synthesized optimized transcription factor binding site
gatccagtactcatgttcattttctctaga

SEQ ID NO: 12 - DNA, 30 bp, Artificial
  synthesized optimized transcription factor binding site
gatccagtactgtttgggaaattccatgga

SEQ ID NO: 13 - DNA, 30 bp, Artificial
  synthesized optimized transcription factor binding site
gatccagtactcccctaattcagacatgca

SEQ ID NO: 14 - DNA, 30 bp, Artificial
  synthesized optimized transcription factor binding site
gatccagtactaataataaaatacccggga

SEQ ID NO: 15 - DNA, 30 bp, Artificial
  synthesized optimized transcription factor binding site
gatccagtactttattataatatgttaaca

SEQ ID NO: 16 - DNA, 30 bp, Artificial
  synthesized optimized transcription factor binding site
gatccagtactgggaaaaaaatcgtcgaca

SEQ ID NO: 17 - DNA, 1189 bp, Homo sapiens
  misc_feature: CEBP rich transcription factor binding site region of MAR 1_68
ttataccaacctcataaaataagagcatatataaaagcaaatgctcttatcttgcagatccctgaactgaggaggcaagatcagtttggcagttgaagcagctggaatctgcaattcagagaatctaagaaaagacaaccctgaagagagagacccagaaacctagcaggagtttctccaaacattcaaggctgagggataaatgttacatgcacagggtgagcctccagaggcttgtccattagcaactgctacagtttcattatctcagggatcacagattgtgctacctattgcctaccatctgaaaacagttgcttcctatatttcatccagtttaatatttatttaaaccaagaaggttaatctggcaccagctattccgttgtgagtggatgtgaaagtaccaattccattctgttttactattaactatcctttgccttaatatgtatcagtaggtggcttgttgctaggaaatattaaatgaatggcatgtttcataggttgtgtttaaagttgttttttgagttaaatctttctttaataatactttctgatgtcaaaaacacttagaagtcatggtgttgaacatctatatagggttggatctaaaatagcttcttaacctttcctaaccactgtttttgtttgtttgtttttaactaagcatccagtttgggaaattctgaattaggggaatcataaaaggtttcattttagctgggccacataaggaaagtaagatatcaaattgtaaaaatcgttaagaacttctatcccatctgaagtgtgggttaggtgcctcttctctgtgctcccttaacatcctattttatctgtatatatatatattcttccaaatatccatgcatgggaaaaaaaatctgatcataaaaatattttaggctgggagtggtggctcacgcctgtaatcccagcactttgggaggctgaggtgggcggatcatgaggtcaagagatcgagaccatcctgaccaatatggtgaaaccccatctctactaaagatacaaaactattagctggacgtggtggcacgtgcctgtagtcccagctactcgggaggctgaggcaggagaacggcttgaacccaggaggtggaggttgcagtgagctgagatcgcgccactgcactccagcctgggcgacagagcgagactctgtctc

SEQ ID NO: 18 - DNA, 763 bp, Homo sapiens
  misc_feature: bent AT/TA dinucleotide rich region of MAR 1_68
aaaaaaaaaatatatatatatatatatatacacatatatatataaaatatatatatatacacacatatatatataaaatatatatatatacacacatatatataaaatatatatatatacacacatatatataaaatatatatatacacacatatatataaaatatatatatacacacatatatataaaatatatatatacacacatatatataaaatatatatatacacacatatatataaaatatatatatacacacatatatataaaatatatatatacacacatatatataaaatatatatatacacacatatatataaaatatatatatacacacatatatataaaatatatatatacacacatatatataaaatatatatatacacacatatataaaatatatatatacacacatatataaaatatatatatacacatatatataaaatatatatatacacatatatataaaatatatatacacacatatatataaaatatatatatacacacatatatataaaatatatatatacacatatatataaaatatatatatacacatatatataaaatatatatatatacacatatatataaaatatatatacacacatatatataaagtatatatatacacacatatatataaaatatatatatacacatatatataaaatatatatatacacatatatataaaatatatatatacacatatatataaaaatatatatatatattttttaaaatattc

SEQ ID NO: 19 - DNA, 1648 bp, Homo sapiens
  misc_feature: Hox-rich transcription factor binding site region of MAR 1_68
caattgtctcactttgtggatgagaaaaagaagtagttagaggtcaagtaacttggcctacatcttttctcaagattgtaaactcctagtgagcaataaccacatcttcattttctttgtataaaacaagaaagtttagcatgaaaaaggtactcaattacaaatgtgttggattgaattgaagacccttggaaggggattttgtacctgaggatctctttcttttggccatattgttcaatggacaaaatttagccttcgaaggcaggccgatttgaggttaatactacctttaccacttgatagctatgtgaccttggccatgtggtttcaacagtctgaacctcattttctctgtgtatgtgtggtcctccttacaagtttgtgaaaaatgtgaagtccttagccatgatagcccaatataacaggctaaatgataataggtttatgttcttttcctttatattctcagataagcactgtccaagtttgaggtgttttgaggtctcgcctgatttggattgtttgagtttatgctattctttgaattctttgagctgttctgaagcagtgtatcatgaacaaaaacatccccagttcagtccaaacccctggttacatatcattcttatgccatgttataaccagtttgagagtgttccctctgttattgcatttaagtttcagcctcacacagaaattcagcagccaatttctaagccctaagcataaaatctggggtgggggggggggatggcctgaagagcagcattatgaatagcaccattataattaatgatctctcaggaagatttacaatcacaggtagcagataaaacaaatagtactgcttctgcacttcccctccttttattcgctatgaaattttatgggaaatcagtccagtgaaaaatgtaagctcttaatctttcccagaaatcctacctcatttgatgaatactttgagggaatgaattagagcatttttttcttttatagtctacttcgcatttacgaagtgaggacggtagcttaggctgcctggccaactgatgagaaggtcagaggcatttttagagacctctgttgtctttcattcatgttcattttccacaaggcaagtaatttccaacaaatcagtgtcttcattagtaataagattattaacaacaataatagtcatagtaactattcagtgagagtccattatatatcaggcattctacaaggtactttatatacatctgagtaaacctcacacaattctacagggaggtatttctatccccatttaacaaataaggaaacgaagtccaagtaaattaacttgcccaaggtcacacagatagtacctggcagaacaggaatttaaacctaaatttgtccaactccaaaagcagccttctatttgttataaatgctgcctctcattatcacatattttattattaacaacaacaaacataccaattagcttaagatacaatacaaccagataatcatgatgacaacagtaattgttatactattataataaaatagatgttttgtatgttactataatcttgaatttgaatagaaatttgcatttctgaaagcatgttcctgtcatctaatatgattctgtatctattaaaatagtactaca

SEQ ID NO: 20 - DNA, 223 bp, Homo sapiens
  misc_feature: 3′ end of Hox-rich transcription factor binding site region of MAR 1_68
agaaagagatcctcaggtacaaaatccccttccaagggtcttcaattcaatccaacacatttgtaattgagtacctttttcatgctaaactttcttgttttatacaaagaaaatgaagatgtggttattgctcactaggagtttacaatcttgagaaaagatgtaggccaagttacttgacctctaactacttctttttctcatccacaaagtgagacaattg

(19) United States
(12) Patent Application Publication
(10) Pub. No.: US 2011/0061117 A1
(43) Pub. Date: Mar. 10, 2011

(54) MATRIX ATTACHMENT REGIONS (MARS) FOR INCREASING TRANSCRIPTION AND USES THEREOF

(75) Inventors: Nicolas Mermod, Buchillon (CH); Pierre Alain Girod, Lausanne (CH); David Calabrese, Perly (CH); Alexandre Regamey, Lausanne (CH); Saline Doninelli-Arope, Lausanne (CH)

(73) Assignee: SELEXIS S.A., Plan-les-ouates (CH)

(21) Appl. No.: 12/438,175

(22) PCT Filed: Aug. 22, 2007
(86) PCT No.: PCT/IB07/02404

§ 371 Date: Oct. 15, 2009

Related U.S. Application Data
(60) Provisional application No. 60/823,319, filed on Aug. 23, 2006.
(60) Provisional application No. 60/953,910, filed on Aug. 3, 2007.

(51) Int. Cl. A01K 67/00; C12N 15/63; C12P 21/00; C07H 21/00; C12Q 1/68; C12N 5/00; C12N 15/00

(52) U.S. Cl. 800/13; 435/320.1; 435/69.1; 536/23.1; 435/6; 435/325; 435/440

(57) ABSTRACT

Isolated and purified MAR sequences of human and non-human animal origin are disclosed as are nucleotide sequences corresponding to or based on them. In particular, MARs and MAR constructs with high transcription and/or protein production enhancing activities are disclosed and so are methods for identifying such MARs, designing such MAR constructs and employing them, e.g., for high yield production of proteins.

### CROSS REFERENCE TO RELATED APPLICATIONS

This application claims the benefit of U.S. provisional application Nos. 60/823,319, filed Aug. 23, 2006 and 60/953,910, filed Aug. 3, 2007, which are incorporated herein by reference in their entirety.

### FIELD OF THE INVENTION

The present invention relates to nucleic acids comprising nucleotide sequences corresponding to or based on isolated and purified MAR sequences of human and non-human animal origin. These nucleic acids generally have transcription and/or protein production enhancing activities. The invention also relates to methods for identifying such sequences and systems employing them, e.g., for high yield production of proteins.

### BACKGROUND

The publications and other materials, including patents, used herein to illustrate the invention and, in particular, to provide additional details respecting the practice are incorporated herein by reference. For convenience, the publications, as far as not stated in full within the text are listed in alphabetical order in the appended bibliography. EMBL accession no. AC102666 and sequences flanked by EMBL accession no. BH101870 and BH101901 as well as EMBL accession nos. (synonyms). 126658, 23119391, 22981746 are also incorporated herein by reference in their entirety.

Nowadays, the model of the organization of eukaryotic chromosomes into chromatin loop domains of about 50 to 100 kb is widely accepted [Bodnar J W, Breyene P, Van Montagu M and Gheyseu G, Razin S V]. The outer ends of these loops are believed to correspond to specific DNA sequences that are attached to the nuclear matrix, a proteinaceous network made up of RNPs (ribonucleoproteins) and other nonhistone proteins [Bode J, Benham C, Knopp A and Mielke C]. The chromosomal DNA sequences that are attached to the nuclear matrix are called SAR or MAR, respectively, for scaffold (during metaphase) or matrix (interphase) attachment regions. S/MARs, MAR elements or MAR sequences or MARs for short, are polymorphic regions of typically 300-3000 bp length. It is estimated that there are approximately 100 000 MARs in a mammalian nucleus [Bode J, Stengert-Iber M, Kay V, Schlake T and Dietz-Pfeilstetter A].

By structurally and functionally segregating the chromatin into looped domains, MAR elements are considered to play a crucial role in the replication and regulation of gene expression such as to facilitate the sequential assembly and disassembly of transcription foci in mammalian nuclei. A host of indirect evidence has been generated to support this notion; for instance, in various eukaryotic genomes, DNA replication origins were mapped within MAR elements [Amati B and Gasser S M (1988), Amati B and Gasser S M (1990)]. MARs are also almost always found in non-coding intergenic regions, within introns [Girod P A, Zahn-Zabal M and Mermod N] or at the borders of transcription units [Gasser S M and Laemmli U K; National Center for Biotechnology Information], where they can bind ubiquitous and/or tissue-specific transcription factors. Overall, in transgenic experiments in plants and in animal cell lines, MAR elements have been successfully used to increase transgene expression and stability [Allen G C, Spiker S, Thompson W F, Bode J, Schlake T, Rios-Ramirez M, Mielke C, Stengart M, Kay V and Klehr-Wirth D, Girod P A, Zahn-Zabal M and Mermod N]. For instance, MARs have been used to increase the production of various recombinant proteins in cells relevant to biotechnology and therapeutic applications, such as CHO (chinese hamster ovary) cells [Girod P A, Zahn-Zabal M and Mermod N, Kim J M, Kim J S, Park D H, Kang H S, Yoon J, Baek K and Yoon Y, Zahn-Zabal M, Kobr M, Girod P A, Imhof M, Chatellard P, de Jesus M, Wurm F and Mermod N] (Mermod et al., “Development of stable cell lines for production or regulated expression using matrix attachment regions,” WO 02074969, also U.S. Patent publication 20030087342).

The functional activity of MARs has been linked to their structural properties rather than to their primary DNA sequence. Indeed, MARs are high in A and T content [Boulikas T (1993)] and some particular conformational and physicochemical properties have been observed, such as a natural curvature of the molecule, a narrow minor groove, a high unwinding/unpairing potential or a susceptibility to denature [Bode J, Schlake T, Rios-Ramirez M, Mielke C, Stengart M, Kay V and Klehr-Wirth D, Boulikas T (1993), Boulikas T (1995)]. In fact those very properties have been used to identify MARs via a method called SMAR Scan. In addition, MAR activity may also be mediated by DNA binding proteins, such as chromatin remodeling enzymes and/or transcription factors that may recognize specific structural features of MAR elements such as single stranded and/or curved DNA [Bode J, Stengert-Iber M, Kay V, Schlake T and Dietz-Pfeilstetter A]. No clear-cut protein-binding site or MAR consensus sequence has been found [Boulikas T (1993)], which makes the prediction of MARs from genomic sequences difficult.

While certain functional and structural properties of MARs have been described, their identification is difficult, since they share little in terms of primary structure. While MAR elements may be functionally conserved in eukaryotic genomes, an assumption which is supported by the fact that animal MARs can bind to plant nuclear scaffolds and vice versa [Breyne P, Van Montagu M, Depicker A and Gheysen G, Mielke C, Kohwi Y, Kohwi-Shigematsu T and Bode J], little can be said about what feature renders a MAR sequence, e.g., a potent protein producing sequence. Also, varying results can be obtained depending on the assay employed [Razin S V, Boulikas T (1995), Kay V and Bode J]. Considering the huge number of expected MARs in an eukaryotic organism and the amount of sequences issued by genome projects, tools/programs were developed to detect the structural features of the MAR DNA sequences (SMAR Scan I), or functional sequences such as the binding sites for specific proteins that act as regulatory proteins or transcription factors (SMAR Scan II) [U.S. provisional patent application 60/953,910, filed Aug. 3, 2007, U.S. Patent Publication 20070178469 to Mermod et al.]. Such programs were designed to identify novel potential MAR sequences by detecting clusters of DNA sequence features corresponding to DNA bending, major groove depth and minor groove width potentials, as well as binding sites for specific transcription regulatory proteins. These programs have been used to scan the human genome to identify putative MAR DNA sequences, several of which were shown to increase transgene expression when introduced into an expression plasmid that was transfected into CHO cells (Girod et al., “Identification of S/MAR from genomic sequences with bioinformatics and use to increase protein production in industrial and therapeutic processes,” U.S. Patent Publication 20070178469 to Mermod et al.]. This demonstrated that the SMAR Scan programs can efficiently identify human genetic elements that, in turn, can be used to increase protein synthesis. While functional screens performed so far were limited to the human genome, in large-scale production, a protein of interest is often expressed in non-human mammalian cells.

About sixteen hundred MARs have been identified in the human genome by SMAR Scan and six out of eight were demonstrated to trigger enhanced expression of genes (such as for green fluorescence protein (GFP), antibodies and receptors) in CHO cells when placed upstream of the enhancer/promoter. The length of DNA shown to have ectopic MAR activity ranges from 2.5 kb to 6 kb. However, the lack of structural characterisation of MARs has, as of now, limited the production of “designer” MARs. Thus, there is a need for the characterization of MARs, in particular functional and/or structural regions of MARs, to allow for MAR engineering and design.

The functional screens performed so far were limited to the human genome. Since in large-scale production, a protein of interest is often expressed in mammalian cells, there is also a need for identifying more potent naturally occurring MARs that enhance transcription and/or gene-expression and/or potent protein producer cells in human and/or non-human mammalian cells.

Overall, a need exists to identify and/or produce MARs having advantageous properties, e.g., by identifying further natural occurring MARs, by engineering identified MARs and/or by producing synthetic MARs. Advantageous properties manifest themselves, but are not limited to enhanced transcription and/or protein production/gene-expression properties; reduced length relative to naturally occurring MARs, thus allowing, e.g., for more versatile use in genetic engineering; tissue, cell or organ specificity and/or inducability upon addition of an external stimulant, such as a drug.

To address one or more of these needs and other needs that will become apparent from the following disclosure, several approaches were employed including a large-scale bioinformatics analysis of the mouse genome to identify putative MAR DNA sequences. The mouse genome was analyzed using MAR predictive software SMAR Scan I. Newly identified rodent sequences were assessed for their ability to mediate improved production of recombinant proteins of pharmaceutical interest from cultured cells. To this end, the transcriptional activity of the newly identified MARs was assessed in transgene transfection assays.

Furthermore, MARs, such as human 1_{—}68 MAR and mouse MAR S4 were studied. Modules, in particular modules comprising certain structural/sequence-specific modules of MARs were identified and these modules utilized to engineer MARs having advantageous properties by, e.g., reshuffling, deletion and/or duplication of sequences. Modules were also combined with other elements, e.g., synthetic nucleotide sequences comprising certain binding sites, in particular transcription factor binding sites (TFBS).

### BRIEF DESCRIPTION OF THE FIGURES

FIG. 1 shows the effect of various MARs on the production of recombinant green fluorescent protein (GFP).

FIG. 2 shows the effect of various human and mouse MAR elements on the percentile of very high producers (% M3) in CHO cells of recombinant green fluorescent protein (GFP).

FIG. 3 shows the effect of various human 1_{—}68 and mouse S4 MAR elements on the expression of recombinant green fluorescent protein (GFP).

FIG. 4 shows the effect of mouse MAR elements on the production of recombinant monoclonal antibodies.

FIG. 5 shows that stable polyclonal populations could be generated from a population of CHO cells transfected with vectors driving expression of IgG heavy and light chains without MAR (no MAR), or with the MAR S4 added in cis.

FIGS. 6 (A) and (B) show that stable individual clones could be generated by limiting dilution from a population of CHO cells transfected with vectors driving expression of IgG heavy and light chains without MAR (no MAR) in (B), or with the MAR S4 and MAR 1_{—}68 added in cis.

FIGS. 7 (A) and (B) shows the expression of a gene (GFP) without a MAR (A) and with a MAR (B) over time (2 weeks and 26 weeks).

FIGS. 8 (A) and (B) depict bending (A) and sequence (B) features of the human 1_{—}68 MAR.

FIG. 9 (A) to (C): (A) show different MAR construct obtained by the shuffling of identified regions and the transcriptional augmentation achieved; (B) shows the bending pattern of MAR construct 6; (C) provide details of structural parameters such as binding sites of the MAR construct 6.

FIG. 10 shows the effect of various MAR S4 constructs on the expression of recombinant green fluorescent protein (GFP) as revealed by the analysis of the average fluorescence of the whole population (Avg Gmean M0).

FIG. 11 shows various MAR S4 constructs derived on the expression of recombinant green fluorescent protein (GFP) as revealed by the analysis of the average fluorescence of the whole population (Avg Gmean M0).

FIG. 12 shows a map of potential transcription factor binding sites of human 1_{—}68 MAR, as predicted by the MATInspector software.

FIG. 13 is a map of the plasmid used to test for the activity of synthetic MARs constructed from the assembly of AT-rich core (MAR 1429-2880) and chemically synthesized DNA binding sites for the transcription factors placed upstream of a promoter and green fluorescent protein (GFP).

FIG. 14 is an illustration of the transcriptional enhancement by synthetic MARs constructed as described in FIG. 13.

FIG. 15 is an illustration of the transcriptional enhancement by synthetic MARs comprising the DNA-binding sites detailed in Table 5.

### SUMMARY OF THE INVENTION

The present invention is, in one embodiment, directed at an expression system for high-level expression of at least one gene comprising:

a promoter for operably liking a nucleotide sequence encoding a gene of interest, and
at least one non-human mammalian MAR nucleotide sequence for enhancing expression of a said gene in a cell transformed with said expression system,
wherein said non-human mammalian MAR nucleotide sequence increases expression of said gene about 2, about 3, about 4, about 5, about 6, about 7, about 8, about 9, about 10 fold or more upon transformation of said cell with said construct.

Said non-human mammalian MAR nucleotide sequence may comprise, consist essentially of or consist of:

(i) SEQ ID No. 3, SEQ ID No. 10 or a functional fragment thereof; or
(ii) a nucleotide sequence having about 80%, about 90%, about 95% or about 98% sequence identity with any of the sequences of (i).

The invention is also directed at an isolated and purified nucleic acid molecule comprising, consisting essentially of or consisting of:

(a) the nucleotide sequence of SEQ ID No. 3 or SEQ ID No. 10 or a functional fragment thereof, or
(b) a nucleotide sequence that has at least about 80%, about 90%, about 95% or about 98% sequence identity with the sequence of (a) and has MAR activity.

The invention is furthermore directed at a method for identifying non-human mammalian MAR sequences comprising:
providing at least one non-human mammalian nucleic acid molecule, preferably a non-human mammalian genome or a part thereof,
subjecting said nucleic acid molecule to a scanning procedure for MAR sequences comprising:
   setting a window size for nucleic acid molecules to be evaluated,
   selecting at least 1 or at least 2, preferably 3, more preferably 4 or more MAR associated features,
   setting threshold values for sequences displaying this/these feature(s), and
   selecting MAR candidate nucleotide sequences exceeding these threshold values,
   ascertaining that said non-human mammalian MAR nucleotide sequence increases expression of a gene about 2, about 3, about 4, about 5, about 6, about 7, about 8, about 9, about 10 fold or more upon transformation of a human and/or non-human mammalian cell via an expression system comprising said non-human mammalian MAR nucleotide sequences.

The feature may hereby be the DNA bending angle whose value is multiplied with the window value to obtain a multiplication value of between about 320 and 1320 such as, about 420 and about 1220, about 520 and about 1120, about 620 and about 1020, about 720 and about 920; the feature may hereby be the major groove depth value which is multiplied with the window value to obtain a multiplication value between about 900 and about 4000, such as about 1200 and 3700, about 1500 and about 3400, about 1800 and about 3100, about 2100 and about 2800 and/or the feature may hereby be minor groove depth value which is multiplied with the window size value to obtain a multiplication value between about 500 and about 2500, such as about 750 and about 2250, about 1000 and about 2000, about 1250 and 1750.

The invention is also directed towards MAR constructs comprising:

(a) (i) an isolated nucleotide sequence comprising at least part of a terminal region of an identified MAR, and
(ii) a further isolated nucleotide sequence comprising about 10%, about 15%, about 20%, about 25%, about 30% or more of said identified MAR or another identified MAR; or
(b) (i) a nucleotide sequence having about 90%, about 95%, about 96%, about 97% about 98%, about 99% sequence identity with the nucleotide sequence of (a) (i), and
(ii) a nucleotide sequence having about 70%, about 80%, preferably about 90%, about 95%, about 96%, about 97% about 98%, about 99% sequence identity with the nucleotide sequence of (b) (i).

Other MAR constructs according to the invention comprise: regions of an identified MAR sequence or a part thereof in consecutive arrangement, wherein an order and/or an orientation differs from that of an identified MAR sequence.

Yet other MAR constructs according to the invention comprise:

(a) a core nucleotide sequence comprising
(i) at least one isolated or synthetic AT-rich region of an identified MAR sequence; or
(ii) at least one AT rich region having at least at least 80%, 85%, 90%, 95%, 98% or 99% sequence identity with the AT-rich region of (a) (i),
   (b) an nucleotide sequence comprising
at least one DNA protein binding site adjacent to said nucleotide sequence of (a), wherein said binding site is
(i) a DNA protein binding site of a further identified MAR sequence,
(ii) a DNA protein binding site of the identified MAR sequence of (a), wherein said DNA protein binding site is, in the identified MAR sequence, situated outside the core nucleotide sequence of (a), or
(iii) a first DNA protein binding site present in the core of (a), but adjacent to at least one further DNA protein binding site, wherein the first and at least one of said further DNA protein binding sites are not adjacent in the core of (a), or
(iv) a DNA protein binding sites of a non-MAR sequence.

The invention is also directed at expression systems comprising any of the specified MAR constructs, kit comprising any of the specified expression systems, and the use of any of the MAR constructs, expression systems, cells, transgenic non-human animals, kits and/or methods referred to herein in (1) producing proteins such as antibodies recognizing human pathogen proteins or human cell surface proteins and proteins such as erythropoietin, interferons or other therapeutic or diagnostic proteins and/or (2) in vitro, in vivo gene therapy, cell therapy or tissue regeneration therapy.

### DETAILED DESCRIPTION OF VARIOUS AND PREFERRED EMBODIMENTS OF THE INVENTION

The present invention relates to isolated and purified MAR sequences from non-human animals, a method of identifying those sequences and a system employing those sequences for the high yield production of proteins in human cells as well as non-human cells such as rodent cells.

The present invention is also directed at MAR constructs, in particular enhanced MAR constructs, expression systems and kits employing these MAR constructs and their use in the production, in particular large scale production of proteins and in therapy. Furthermore, the invention is directed at methods for the high yield production of proteins in human cells as well as non-human mammalian cells via MARs/MAR constructs.

Unless otherwise defined, all technical and scientific terms used herein have the same meaning as commonly understood by one of ordinary skill in the art to which this invention pertains. Although methods and materials varying from those described herein can be used in the practice of the present invention, examplaratory suitable methods and materials are described below.

An expression cassette according to the present invention is a nucleic acid comprising at least one gene as well as elements required for the transcription of this gene.

A promoter according to the present invention is regulatory region of DNA, that, when located upstream of a gene, furthers transcription of the gene.

Expression in a cell, e.g., expression in a non-human mammalian cell, refers, in the context of the present invention, to expression in vitro and in vivo. In vitro expression includes, e.g., expression in a cell line such as a HeLa cell line or a CHO cell line and in cells used for in vitro gene therapy. In vivo expression comprises expression in a transgenic non-human animal and expression in human cells used in vivo gene therapy or in vitro gene therapy after reintroduction of the cells into a human gene therapy recipient.

A mammalian cell, such as a non-human mammalian cell, according to the present invention is capable of being maintained under cell culture conditions. A non-limiting example of this type of cells are chinese hamster ovary (CHOs) cells.

A MAR construct, MAR element, a MAR sequence, a S/MAR or just a MAR according to the present invention is a nucleotide sequence sharing one or more (such as two, three or four) characteristics with a naturally occurring “SAR” or “MAR” and having at least one property that facilitates protein expression of any gene influenced by said MAR. A MAR construct has also the feature of being an isolated and/or purified nucleic acid with MAR activity, in particular, with transcription modulation, preferably enhancement activity, but also with, e.g., expression stabilization activity and/or other activities which are also described under “enhanced MAR constructs.” MAR constructs may be defined based on the identified MAR they are primarily based on: A MAR S4 construct is, accordingly, a MAR construct that whose majority of nucleotide (50% plus) are based on MAR S4. Naturally occurring SARs or MARs, according to a well-accepted model, mediate the anchorage of specific DNA sequences to the nuclear matrix, generating chromatin loop domains that extend outwards from the heterochromatin cores. While SARs or MARs do not contain any obvious consensus or recognizable sequence, their most consistent feature appears to be an overall high A and T content, and C bases predominating on one strand. MARs have generally the propensity to form bent secondary structures that may be prone to strand separation. Several simple sequence motifs high in A and T content have often been found within SARs and/or MARs, but for the most part, their functional importance and potential mode of action has been unresolved. These include the A-box, the T-box, DNA unwinding motifs, SATB1 binding sites (H-box, A/T/C25) and consensus topoisomerase II sites for vertebrates or Drosophila.

A MAR candidate or MAR candidate sequence according to the present invention is a sequence sharing one or more characteristics such as two, three or four with naturally occurring SARs or MARs.

An identified MAR or identified MAR sequence according to the present invention is and isolated nucleotide sequence and corresponds to a naturally occurring MAR sequence in that it comprises all regions (“modules” or “elements”) that allow for the full enhancement of protein/gene expression of its natural counterpart.

The modules (also referred to herein as “regions,” “DNA region”, “portions”, “domains”) of an identified MAR are all required to allow enhancement of protein/gene expression to the capacity of the naturally occurring MAR. None of the modules is generally able to achieve the full activity of the MAR by itself. Some of these regions are sequence specific, such as AT-dinucleotide rich bent regions and transcription factor binding site (TFBS) regions described below. Others “regions” are characterized by their location, e.g., the 5′ and 3′ terminal regions of an identified MAR sequence.

An AT/TA-dinucleotide rich bent DNA region (hereinafter referred to as “AT-rich region”) is a bent DNA region comprising a high number of A and Ts, in particular in form of the dinucleotides AT and TA. In a preferred embodiment, it contains at least 10% of dinucleotide TA, and/or at least 12% of dinucleotide AT on a stretch of 100 contiguous base pairs, preferably at least 33% of dinucleotide TA, and/or at least 33% of dinucleotide AT on a stretch of 100 contiguous base pairs (or on a respective shorter stretch when the AT-rich region is of shorter length), while having a bent secondary structure. However, the “AT-rich regions” may be as short as about 30 nucleotides or less, but is preferably about 50 nucleotides, about 75 nucleotides, about 100 nucleotides, about 150, about 200, about 250, about 300, about 350 or about 400 nucleotides long or longer.

As will be discussed below, an AT-rich region can be distinguished from a neighboring region, such as a binding site region by, e.g., its relative high bending angle. Some binding sites are also often have relatively high A and T content such as the SATB1 binding sites (H-box, A/T/C25) and consensus Topoisomerase II sites for vertebratesor Drosophila. However, a binding site region (module), in particular a TFBS region, which comprises a cluster of binding sites, can be readily distinguished from AT and TA dinucleotides rich regions (“AT-rich regions”) from binding sites high in A and T content by a comparison of the bending pattern of the regions. For example, for human MAR 1_{—}68, the latter might have an average degree of curvature exceeding about 3.8 or about 4.0, while a TFBS region might have an average degree of curvature below about 3.5 or about 3.3. Regions of an identified MAR can also be ascertained by alternative means, such as, but not limited to, relative melting temperatures, as described elsewhere herein. However, such values are species specific and thus may vary from species to species, and may, e.g., be lower. Thus, the respective AT and TA dinucleotides rich regions may have lower degrees of curvature such as from about 3.2 to about 3.4 or from about 3.4 to about 3.6 or from about 3.6 to about 3.8, and the TFBS regions may have proportionally lower degrees of curvatures, such a below about 2.7, below about 2.9, below about 3.1, below about 3.3. In SMAR Scan II, respectively lower window sizes will be selected by the skilled artisan.

A terminal region of an identified MAR/MAR sequence according to the present invention comprises at least about 5%, about 6%, about 7%, about 8%, about 9% or about 10% of an identified MAR.

A binding site or DNA protein binding site is any nucleotide sequence that can bind a DNA binding protein. Binding sites for DNA binding proteins are typically TFBSs. A TFBS is any sequence that can bind a transcription factor. The TFBS can be of any origin such as, but not limited to, human or mouse. TFBSs may also be engineered or synthetic. However, in certain embodiments, the TFBS has a counterpart in a MAR sequence, such as a MAR sequence of the same organism, the same species or the same genus. However, the TFBS may be from a MAR sequence of a different species or a different genus. Also TFBSs that have no currently known counterpart in a MAR sequence are within the scope of the present invention. Such TFBSs may include, but are not limited to, binding sites for USF1 (upstream stimulatory factor 1) or the zink-finger protein CTCF. TFBSs might be modified by 1, 2, 3, 4, 5 or more substitutions, additions and/or deletions and may be in full or part synthesized. Optimized TFBSs, that are TFBSs with optimized binding affinities for the respective DNA binding protein and which often have no known natural counterpart, are also within the scope of present invention. Those optimized TFBS might be created by the above modifications of a natural occurring TFBSs or synthetically, in particular by chemical synthesis. In certain embodiments of the invention, the binding site(s) or TFBS(s) confer tissue specificity to the MAR by, e.g., being bound by tissue-specific natural, engineered or synthetic regulatory proteins or other natural, engineered or synthetic proteins, which, e.g., may respond to specific drugs and molecules. Gene and/or cell therapy are typical cases benefiting from tissue-specificity as well as from the ability of the MAR to specifically respond to a certain drug, that is, be inducible by the drug. In the former case, the, e.g., gene of interest would only be expressed in specific organs or tissues, in the latter case, the expression could, e.g., only be turned on in response to a certain drug. Other non-limiting examples of transcription factors for which TFBSs may be included are, e.g., SATB1, NMP4, MEF2, S8, DLX1, FREAC7, BRN2, GATA 1/3, TATA, Bright, MSX, AP1, C/EBP, CREBP1, FOX, Freac7, HFH1, HNF3alpha, Nk×25, POU3F2, Pitt, TTF1, XFD1, AR, C/EBPgamma, Cdc5, FOXD3, HFH3, HNF3 beta, MRF2, Oct1, POU6F1, SRF, V$MTATA_B, XFD2, Bach2, CDP CR3, Cdx2, FOXJ2, HFL, HP1, Myc, PBX, Pax3, TEF, VBP, XFD3, Brn2, COMP1, Evil, FOXP3, GATA4, HFN1, Lhx3, NKX3A, POU1F1, Pax6 and/or TFIIA.

A binding site, such as a TFBS, is said to be adjacent to a core nucleotide sequence if the core nucleotide sequence and the binding site is separated by not more than about 200, preferably not more than about 100 nucleotides, even more preferably not more than about 50 nucleotides, even more preferably not more than about 25, not more than about 15, not more than about 5 or no nucleotides. In a preferred embodiment the binding site, in particular TFBSs, themselves comprise short linker or adapters of up to 25 nucleotides on each side of the TFBS. In an even more preferred embodiment the TFBS is part of an oligomer of up to about 50 nucleotides, up to about 40 nucleotides or up to about 30 nucleotides. A series of binding sites, such as TFBSs in accordance with the present invention, are a row of TFBSs are arranged in sequence next to each other. A series of TFBSs is said to be adjacent to a core nucleotide sequence if the TFBS of this series which is proximate to the core has the distance specified above. A binding site is said to flank an “AT-rich region” if the binding site is a binding site which is part of the core nucleotide sequence and has a counterpart at the identical location in a naturally occurring MAR.

A binding site may be modified by 1, 2, 3, 4, 5 or more substitutions, additions and/or deletions. Preferably these substitutions, additions and/or deletions are introduced so that the binding site matches a consensus sequence of the respective binding site.

A variety of enhanced MAR construct are part of the present invention and have properties that constitute an enhancement over a naturally occurring and/or identified MAR on which a MAR construct according to the present invention may be based, in particular the natural occurring MAR on which the core nucleic acid sequence is based. Such properties include, but are not limited to, reduced length relative to the full length natural occurring and/or identified MAR, gene expression/transcription enhancement, enhancement of stability of expression, tissue specificity, inducibility or a combination thereof. Accordingly, a MAR construct that is enhanced may, e.g., comprise less than about 90%, preferably less than about 80%, even more preferably less than about 70%, less than about 60%, or less than about 50% of the number of nucleotides of an identified MAR sequence. A MAR construct may enhance gene expression and/or transcription of a gene upon transformation of an appropriate cell with said construct. If, in the context of the present invention, reference is made to MAR constructs/MAR (nucleotide) sequences that “enhance expression,” have a “gene expression enhancing activity,” “enhance protein expression” or similar, this “enhancement” is relative to the expression of, e.g., a gene, expressed under otherwise equivalent conditions but in absence of such a sequence. The enhancement can, for example, be about 2, about 3, about 4, about 5, about 6, about 7, about 8, about 9, about 10 fold or about 15 fold, about 20 fold or about 25 fold or higher.

A MAR construct may also increase the average percentile of very high producing cells by about 5 fold, about 10 fold, about 15 fold or more. Thus, apart from an higher average expression of a gene, an increase in the percentile of very high expressing cells, as well as the occurrence of stable (“resistant”) colonies (about 100%, about 200%, about 300% or about 400% or higher increase, and/or a lower variability of expression (reduction of cv (coefficient of variation) of about 30%, about 40%, about 50% or more) are within the scope of the present invention.

A MAR construct or similar may “enhance stability of expression.” This “enhancement” is relative to the expression of, e.g., a gene being expressed under otherwise equivalent conditions, but in absence of such a MAR construct/MAR sequence. The stability enhancement can, for example, maintain 100% enhancement after up to about 5, 10, 20, 25, 30, 35, 40, 45, or 50 weeks. A MAR construct may by specific for, e.g., muscle, liver, central nervous system or other tissues and/or may be inducible upon administration of a substance such as antibiotics, hormones and/or metabolic intermediates.

A MAR construct/MAR sequence may be inserted preferably upstream of a promoter region to which a gene of interest is or can be operably linked. However, in certain embodiments, it is advantageous that a MAR construct is located upstream as well as downstream or just downstream of a gene/nucleotide acid sequence of interest. Other multiple MAR arrangements both in cis and/or in trans are also within the scope of the present invention.

A MAR construct or a region of a MAR is said to be based on, e.g., an identified MAR or a region of a identified MAR if it shares one or more (such as two, three or four) characteristics with naturally occurring “SARs” or “MARs” or an respective region thereof and has at least one property that facilitates protein expression of any gene influenced by said MAR. These MAR constructs or regions of a MAR generally have “substantial identity” with the identified MARs they are based on in accordance with the definition of the term provided herein. Despite these and/or modifications of their nucleotide sequence, they will maintain at least one functionality/characteristic of the underlying identified MAR.

The present invention is also directed to uses of a MAR constructs, including enhanced MAR constructs. In these uses, a MAR construct may also be combined with one or more non-MAR epigenic gene regulation tool such as, but not limited to, histone modifiers such as histone deacetylase (HDAC), other DNA elements such as locus control regions (LCRs), insulators such as cHS4 or antirepressor elements (e.g., stabilizer and antirepressor elements (STAR or UCOE elements) or hot spots (Kwaks THJ and Otte AP).

Synthetic, when used in the context of a MAR/MAR construct refers to a MAR whose design involved more than simple reshuffling, duplication and/or deletion of sequences/regions or partial regions, of identified MARs or MARs based thereon. In particular, synthetic MARs/MAR constructs generally comprise one or more, preferably one, region of an identified MAR, which, however, might in certain embodiment be synthesized or modified, as well as specifically designed, well characterized elements, such as a single or a series of TFBSs, which are, in a preferred embodiment, produced synthetically. These designer elements are in many embodiments relatively short, in particular, they are generally not more than about 300 bps long, preferably not more than about 100, about 50, about 40, about 30, about 20 or about 10 bps long. These elements may, in certain embodiments, be multimerized.

A non-human mammalian MAR according to the present invention is a MAR/MAR sequence that is, at least in part, ascertained via the genome or parts of the genome of an non-human mammalian organism. This includes, for example MAR/MAR sequences identified via analysis of a rodent genome such as, but not limited to, a mouse genome.

A vector according to the present invention is a nucleic acid molecule capable of transporting another nucleic acid molecule to which it has been linked. For example, a plasmid is a type of vector, a retrovirus or lentivirus is another type of vector.

Transfection according to the present invention is the introduction of a nucleic acid into a recipient eukaryotic cell, such as, but not limited to, by electroporation, lipofection, via a viral vector or via chemical means.

Transformation as used herein, refers to modifying an eukaryotic cell by the addition of a nucleic acid. For example, transforming a cell could include transfecting the cell with nucleic acid, such as by introducing an DNA vector via electroporation. However, in many embodiments of the invention, the way of introducing the enhanced MARs of the present invention into a cell, is not limited to any particular method.

Transcription means the synthesis of RNA from a DNA template.

Cis refers to the placement of two or more elements (such as chromatin elements) on the same nucleic acid molecule such as, but not limited to, the same vector or chromosome.

Trans refers to the placement of two or more elements (such as chromatin elements) on the two or more nucleic acid molecules such as, but not limited to, two or more vectors or chromosomes.

A sequence is said to act in cis and/or trans on, e.g., a gene when it exerts its activity from a cis/trans location.

A window according to the present invention describes a number of base pairs evaluated for MARs, e.g., during the SMAR Scan procedure. The number is usually about 50 bps, about 100 bps, about 200 bps, about 300 bps. However, windows of 400, 500, 600 or more bps are also within scope of the present invention.

A nucleotide sequence or fragment thereof has substantial identity with another if, when optimally aligned (with appropriate nucleotide insertions or deletions) with the other nucleotide sequence (or its complementary strand), there is nucleotide sequence identity in at least about 60% of the nucleotide bases, usually at least about 70%, more usually at least about 80%, preferably at least about 90%, and more preferably at least about 95-98% of the nucleotide bases.

Identity means the degree of sequence relatedness between two nucleotide sequences as determined by the identity of the match between two strings of such sequences, such as the full and complete sequence. Identity can be readily calculated. While there exists a number of methods to measure identity between two nucleotide sequences, the term “identity” is well known to skilled artisans (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). Methods commonly employed to determine identity between two sequences include, but are not limited to those disclosed in Guide to Huge Computers, Martin J. Bishop, ed., Academic Press, San Diego, 1994, and Carillo, H., and Lipman, D., SIAM J Applied Math. 48: 1073 (1988). Preferred methods to determine identity are designed to give the largest match between the two sequences tested. Such methods are codified in computer programs. Preferred computer program methods to determine identity between two sequences include, but are not limited to, GCG (Genetics Computer Group, Madison Wis.) program package (Devereux, J., et al., Nucleic Acids Research 12(1). 387 (1984)), BLASTP, BLASTN, FASTA (Altschul et al. (1990); Altschul et al. (1997)). The well-known Smith Waterman algorithm may also be used to determine identity.

As an illustration, by a nucleic acid comprising a nucleotide sequence having at least, for example, 95% “identity” with a reference nucleotide sequence means that the nucleotide sequence of the nucleic acid is identical to the reference sequence except that the nucleotide sequence may include up to five point mutations per each 100 nucleotides of the reference nucleotide sequence. In other words, to obtain a nucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

Functional fragments of nucleotide sequences are also part of the present invention. A fragment is considered functional as long as they maintain a desirable function of the naturally occurring counterpart sequences, in particular increasing expression of a gene influenced by them. A fragment of a MAR or a MAR region is still considered a functional fragment if it's deletion decreases the transcription enhancing activity of a MAR/region, but does not abolish it. A “fully functional fragment” is a fragment in which any decrease in activity, if at all observed, cannot be statistically verified when the fragment is used without other MAR sequences. Also included within the scope of the present invention are functional fragments having substantial identity in accordance with the definition provided herein with, e.g., the naturally occurring MAR, identified MAR, MAR region or a fragment of any of these.

As will be described in detail herein, in certain embodiments, modules or parts thereof are reshuffled, duplicated and/or subject to deletion. As the person skilled in the art will recognize, such, shuffling and/or duplication of regions, may create, e.g., new restrictions sites, which in turn can lead to new restriction pattern of the constructs so created and may lead to adjustments in the length of the sequences. Those adjustments may affect, but are not limited to, 1, 2, 3, 4, 5, 6, 7, 8, 9, 10, 10-15, 15-20, 20-25, 25-30, 30-35, 35-40 nucleotides. These adjustments as well as other modifications are within the scope of the present invention. Sequences of the rearranged MARs, in particular reshuffled and/or duplicated MARs, that have substantial identity in accordance with the definition provided herein with each of the respective element(s) (or region(s)/module(s)) and/or fragment(s) thereof, are within the scope of the present invention.

MAR sequences can be transferred from plant to mammalian cells or vice versa, and will retain nuclear matrix attachment activity in the heterologous host cells [Breyne P, Van Montagu M, Depicker A and Gheysen G, Mielke C, Kohwi Y, Kohwi-Shigematsu T and Bode J]. Given this conservation of MAR functions in all higher eukaryotes, one would expect that a MAR sequence from one genus would work as well in the genus it was derived from as in another genus.

Nonetheless, reasoning that MAR sequences from rodent origins might be in some way advantageous for the production of recombinant proteins, the whole mouse genome was screened to identify MAR candidate sequences using SMAR Scan I, a computer program that, as described below, detects structural features of the DNA sequences (DNA bend, for example).

As will be discussed below, it was surprisingly found that non-human, in particular rodent (here mouse) MAR sequences are more potent in terms of expression enhancement, e.g., in CHO cells as well as human cells such as HeLA cells. Even more surprisingly, it was found that certain non-human MAR sequences work substantially better, both in non-human cells, e.g., CHO cells as well as in human cells, e.g. in HeLa cells, than human MAR sequences.

Several of the identified novel S/MAR DNA sequences of mouse origin were could be shown to increase transgene expression, thus providing evidence that SMAR Scan I, a program designed for and tested with human MAR sequences, is an efficient tool for identifying S/MAR elements from a multitude of genomic origins, e.g., mouse in addition to human. Importantly, however, it was found that more potent MAR elements can be identified by screening rodent (e.g., mouse) genomes than by screening the human genome. In particular, the invention establishes that highly active S/MAR elements from the mouse genome can be used to increase the production of recombinant proteins, such as recombinant proteins having pharmaceutical uses, in a variety of cells, in particular mouse and human cells. The mouse S/MAR S4 was shown to be the most potent of the newly isolated mouse MARs and of the previously cloned human MARs. The invention is thus directed at non-human MARs having enhanced protein production and/or at MARs enhancing the stability of protein expression over time.

SMAR Scan I is a software tool that identifies MAR candidate sequences based on the structural and physicochemical features of these sequences. A thorough discussion of the method has been provided elsewhere (U.S. Patent Publication 20070178469 to Mermod et al). Essentially, “SMAR Scan” describes bioinformatic tools comprising algorithms that recognize profiles, based on dinucleotide weight-matrices, to compute the theoretical values for conformational and physiochemical properties of DNA. Preferably, SMAR Scan evaluates DNA sequence features corresponding to DNA bending, major groove depth and minor groove width potentials, melting temperatures in a wide variety of combinations using scanning windows of variable sizes. For each feature, a cut-off or threshold value has to be set. The program returns a hit each time the computed score of a given region is above the set cut-off/threshold value.

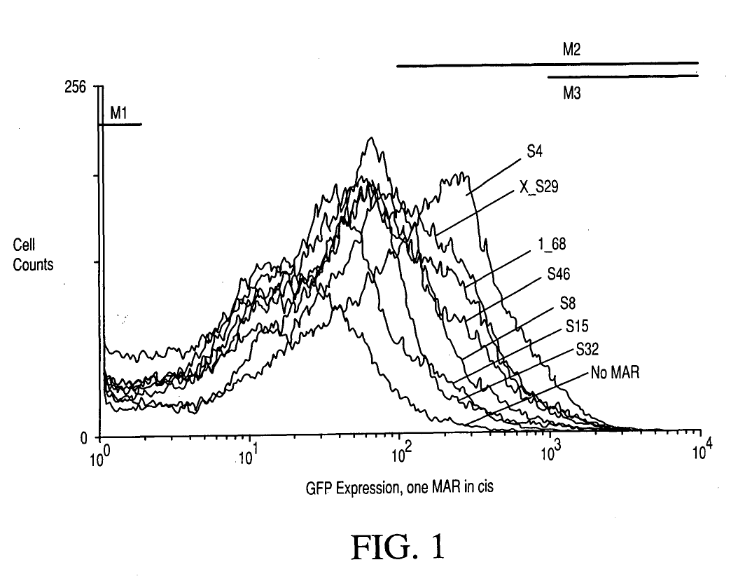

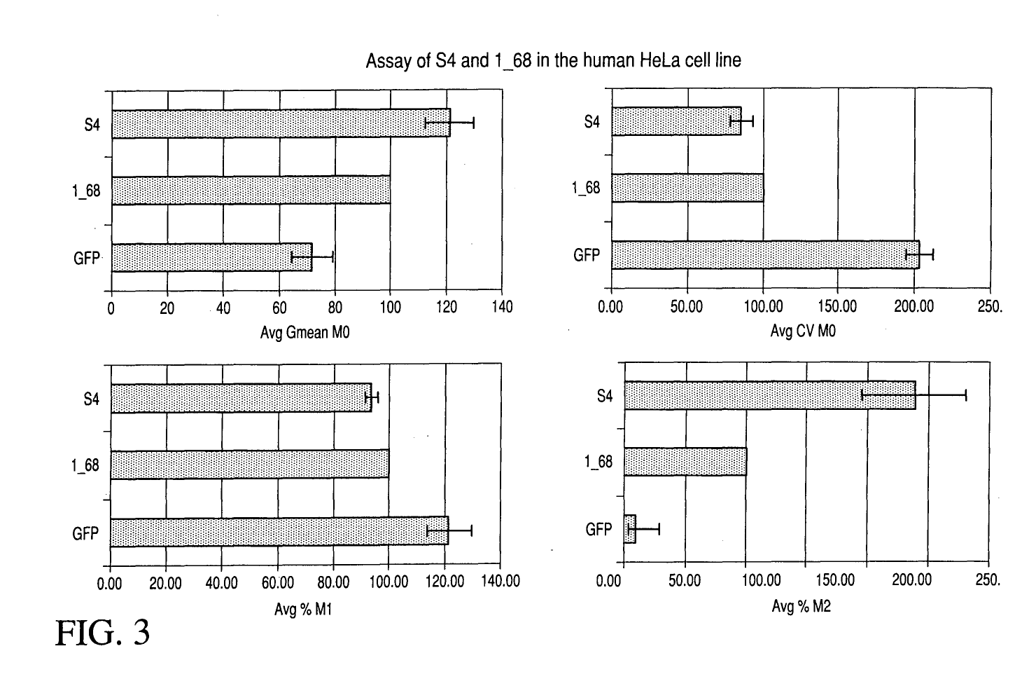

Two data output modes are available to handle the hits, the first (called “profile-like”) simply returns all hit positions on the query sequence and their corresponding values for the different criteria chosen. The second mode (called “contiguous hits”) returns only the positions of several contiguous hits and their corresponding sequence. For this mode, the minimum number of contiguous hits is another cut-off/threshold value that can be set, again with a tunable window size. To tune the default cut-off/threshold values for, e.g., the four theoretical structural criteria, experimentally validated MARs, e.g., from SMARt DB can be used. In this way, for example, all human MAR sequences from the database were retrieved and analyzed with SMAR Scan using the“profile-like” mode with the four criteria and with no set cut-off/threshold value. This allowed the setting of each function for every position of the sequences. The distribution for each criterion was then computed according to these data (see FIGS. 1 and 3 U.S. Patent Publication 20070178469 to Mermod et al).

While the use of SMAR Scan technology is a preferred one for the identification of MAR sequences, the person skilled in the art will recognize that other bioinformatic tools that allow for the identification of S/MAR motives with similar or even somewhat lower selectivity can be used in the context of the present invention. Preferably such tools can be set so that only those MAR associated features that display these features beyond a certain value, that is a set threshold or cut-off value, yield or can be set to yield a positive hit. Many bioinformatic tools used to identify MARs were, however, designed to identify matrix-binding activity. This activity does not necessarily correlate with the ability to increase gene expression [Phi-Van, L. & Stratling, W. H.].

SMAR Scan I has been developed to identify human MARs. Thus, it was developed using structural data collected from known human MARs. A human “tuned” SMAR Scan I program was used in context of the present invention to evaluate the mouse genome for MAR sequences. However, differences in the base compositions of the mouse and human genomes prevented the use of SMAR Scan program with the settings previously defined to scan the human genome (U.S. Patent Publication 20070178469 to Mermod et al). Therefore distinct window size and structural parameter threshold values had to be defined by trial and error, until the program would allow the identification of a manageable collection of candidate mouse MAR sequences. Several of those, when tested, turned out to be “super MAR sequences”, that are MAR sequences allowing for substantial increase of protein production, when, e.g., placed on a vector with the gene encoding the respective protein and introduced into a rodent cell line.

Mouse MAR S4 and Mouse MAR S46 are examples of rodent MAR sequences that are within the scope of the present invention. These MAR sequences as isolated are shown in the appended sequence listing as SEQ ID No. 3 and SEQ ID No. 10. However, as the person skilled in the art will appreciate, base pair insertions, deletions, substitutions, in particular fragments of these and other non-human MARs that themselves may contain base pair insertions, deletions or substitutions are within the scope of the present invention as long as they maintain a desirable function of the wild type sequences, in particular increasing expression of a gene influenced by them. For example, an insertion that decreases the transcription/gene expression enhancing activity of a MAR sequence, but does not abolish it, is considered to not substantially interfere with the desirable function, here gene expression enhancement, of the MAR. Similarly, a fragment of an, e.g., identified MAR is still considered a functional fragment if has a somewhat reduced transcription enhancing activity relative to the identified MAR, but does not completely lose the transcription enhancing activity. A “fully functional fragment” is a fragment in which any decrease in activity, if at all observed, cannot be statistically verified. As detailed elsewhere herein, also included within the scope of the present invention are sequences having “substantial identity” with the nucleotide sequence of the naturally occurring MAR or a fragment thereof.

### Modularity of MARs

Identified MARs were analyzed to determine whether they comprise modules (or regions), in particular sequence-specific modules, which could be used in engineering identified MARs or in producing synthetic MARs, including MARs comprising synthesized regions. In fact, several sequence-specific modules of identified MARs could ascertained. Surprisingly it was found that shuffling and/or full or partial duplication and even deletion of certain modules or parts thereof resulted in enhanced MARs as described above.

The human 1_{—}68 MAR and S4 MAR from mouse will serve as a model for producing MAR constructs by shuffling, deleting and/or duplication of regions. However, as the person skilled in the art will readily understand, the present invention is directed at manipulating any identified MAR and at the MAR constructs resulting therefrom. Appropriate adjustments that may be necessary to accommodate different MARs, including MARs of different origin, are well within the skill of the artesian. Examples include, but are not limited to, eukaryotic organisms, preferably mammals, especially model organisms such as mouse, and species of economic importance such as cattle, pigs, sheep as well as humans.

### Modularity of Human MARs

The human 1_{—}68 MAR served as a model for producing MAR constructs by shuffling and/or duplication of regions. Using modules ascertained as described below or parts thereof, MAR constructs were produced based on identified MARs, such as human 1_{—}68 MAR. The MAR constructs were in particular produced by shuffling, and/or duplication of regions (modules) or parts thereof.

The 1_{—}68 MAR example shows that modules (also referred to herein as regions or elements) of an identified MAR were all required to allow enhancement of gene expression to the capacity of the naturally occurring MAR. None of the modules identified was able to achieve the full activity of the MAR by itself. Surprisingly, it was found that shuffling and full or partial duplication of certain modules resulted in further enhancement of gene expression.

Several non-redundant sequence-specific modules (regions) were identified. These modules cooperate to influence local chromatin structure. This organization of MAR parallels somewhat the control of metazoan transcription: a diverse collection of modules, which are dispersed up to several kilobases from the initiation site, collectively dictate where transcription will initiate.

The sequence-specific modules identified were in particular (1) regions high in A and T content, such as symmetrical A-T rich regions (alternating A and T) in particular “AT rich regions” and (2) regions rich in binding sites, in particular, but not limited to, TFBSs separated by A-T rich regions.

It has been reported that bent DNA high in A and T content are commonly found in promoter regions, MARs and replicators [Aladjem and Fanning 2004]). Previously, sequences high in A and T content (“symmetric” ones as described above as well as “asymmetric” ones, that are sequences having mostly A on one strand and mostly T on the other) were thought to primarily facilitate duplex opening. However, these regions might have a wide range of functions. For example, sequences high in A and T content in the lamin B2 replicator bind the origin-recognition complex (ORC) [Abdurashidova, Danailov et al. 2003; Stefanovic, Stanojcic et al. 2003] and can facilitate the loading of the Mcm4/6/7 helicase and the unwinding of duplex DNA in vitro [You, Ishimi et al. 2003]. Architectural roles for intrinsically bent DNAs high in A and T content have also been considered. The “AT-hook DNA-binding motifs” of fission yeast ORC4, which resemble those of the high mobility group protein HMG-I/Y, may have such an architectural role [Strick and Laemmli 1995; Bell 2002]. Protein-mediated bending, analogous to the HMG-I/Y-mediated DNA bending that facilitates V(D)J recombination, and the assembly and stabilization of transcription complexes at enhancers and promoters in eukaryotes, might also occur [Levine and Tjian 2003]. Not all regions that have a high A and T content correspond to bent DNA. However, those DNAs are bent could act as a ‘histone magnet’ to attract histones to form nucleosomes over the bent DNA, leaving the adjacent regions free to act as a landing pad for pre-replication/transcription proteins.

As described above, MARs also contain binding sites for other proteins in particular in the “regions rich in binding sites” or just “binding site regions” (see (2) above), Those other proteins may include, but are not limited to, DNA unwinding element-binding protein (DUE-B) and transcription factors such as Hox proteins, SATBI, CEBP, etc as found in 1_{—}68 MAR. Mutational analysis indicates that these binding sites contribute to the MAR function.

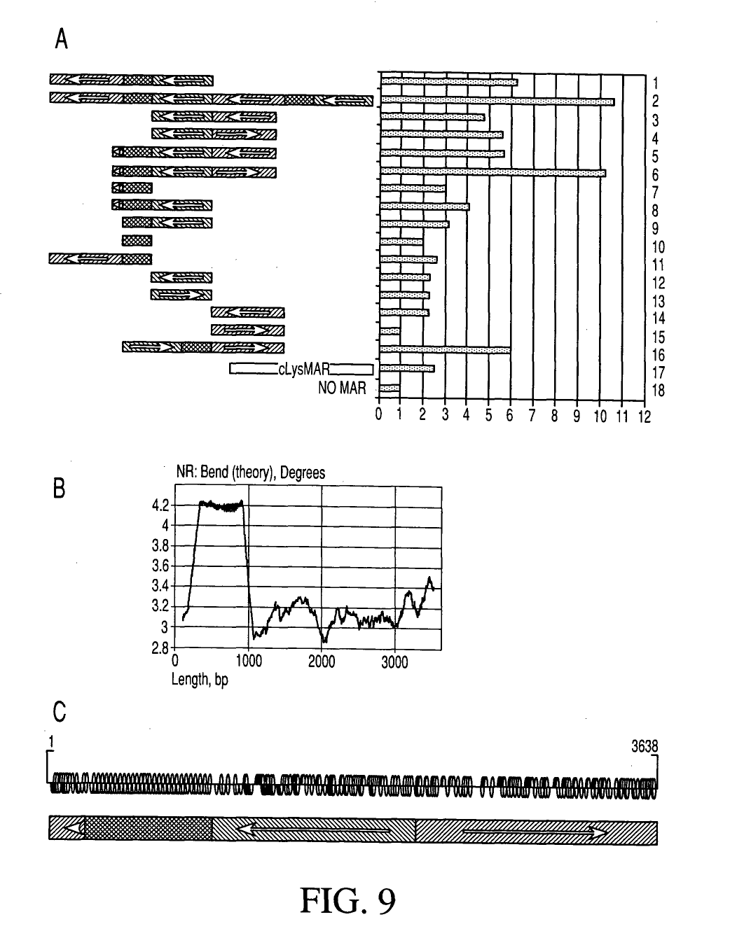

Human 1_{—}68 MAR could be improved by reversing its orientation and by moving away the bent DNA to augment the size of the transcription factors binding site region upstream the promoter region. As can be seen in FIG. 9, a number of these rearranged MARs (e.g. construct 6) considerably augment transcription relative to a construct without MAR (10 fold) and even relative to a construct including the natural occurring MAR (constructs 1 and 16; about 2 fold). The data shown also strongly indicate that a distal transcriptional control element itself restricts transcription initiation in the downstream chromatin. A 223 bp fragment located at the 3′ end of the region shown as a forward hatched box in the naturally occurring MAR retains all the activity of this region in construct 7 as compared to construct 11. This suggests that this important portion must, in this case, cooperate with the bent region and the 5′-end of the remainder (nucleotides 1-1425) of the element in construct 6. Two HMG-I/Y sites were found to be located nearby this terminus. Construct 2 shows that joining two identified MAR sequences together also increases expression.

### Modularity of Mouse MARs and Reduction of Size

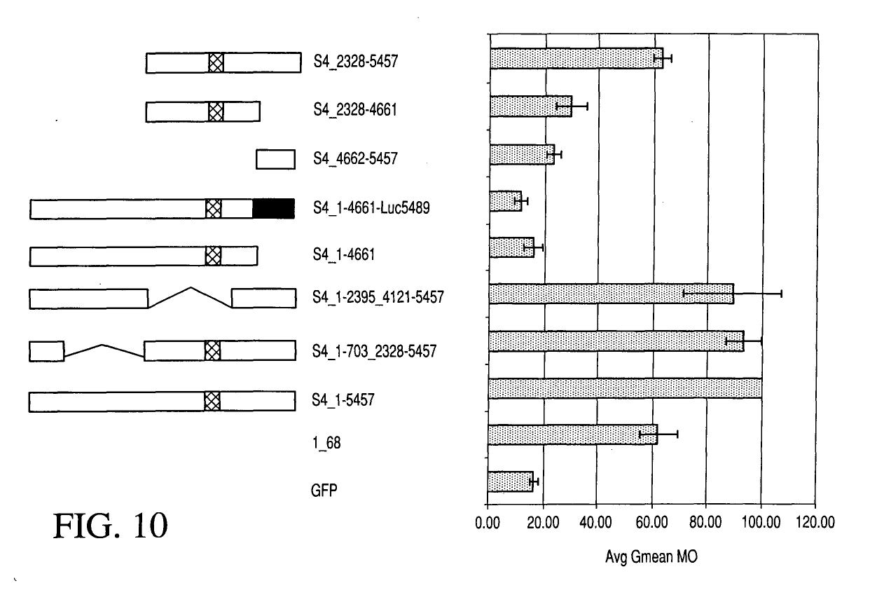

Several MARs were constructed based the S4 MAR (Table 3) and characterized (FIG. 10). As can been seen in FIG. 10, internal deletion of a fragment more than 1600 bps long did not lead to a considerable loss in MAR activity (S4-1-703_{—}2328-5457). However, deletion of the promoter-proximal 795-bp fragment, or replacement of this sequence by a fragment of the luciferase gene of similar length (S4_{—}1-4661; S4_{—}1-4661-Luc5489), induced a complete loss of this activity.

### Non-Sequence Specific Modules: Activity of the 3′ Terminal MAR Sequences

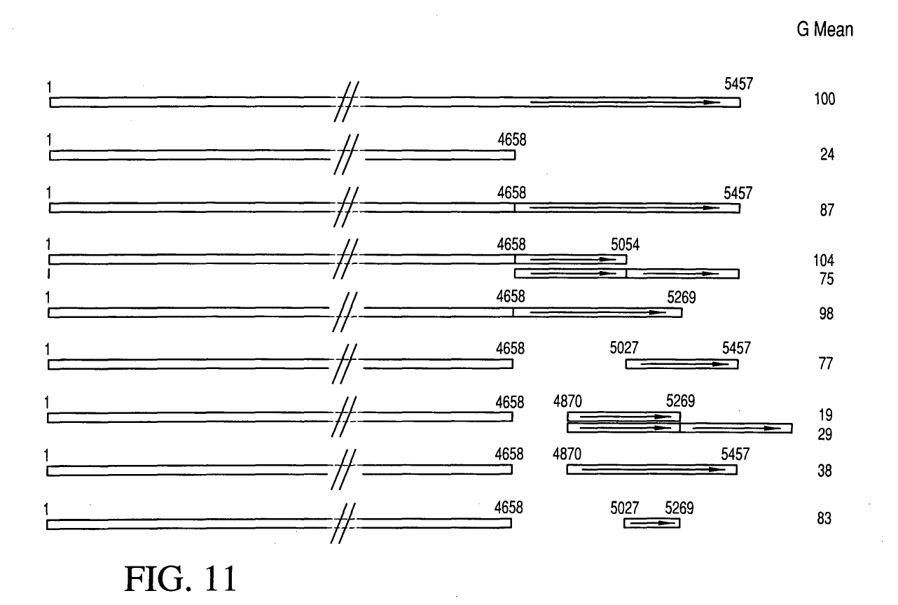

Experiment with the human 1_{—}68 MAR (FIG. 9) already showed the significance of the 3′ HoxF and SATBI binding site region of the human 1-68 MAR. The significance of this region was further manifested by the experiments with mouse MAR S4 shown in FIG. 10. As shown in FIG. 11, to further analyze the activity of the 3′ end sequences of MAR S4, this portion of the MAR was further dissected by removing or duplicating portions of it. FIG. 11 also shows the effect of various MAR S4 derivatives on gene expression. Interestingly, one such derivative, having a truncated 3′ end (4658-5054 vs. 4658-5457 of the original MAR S4), displayed, on average, a slightly higher transgene expression compared to the longer original MAR S4 sequence (104% vs 100%). This indicates that more potent as well as shorter derivatives of MAR elements can be obtained.

Thus, the present invention includes high activity MAR constructs that are considerably shorter in length than their natural counterparts, thus making them of more convenient size for, e.g., vector design and transfer.

In particular, MAR constructs comprising less than about 90%, preferably at less than about 80%, even more preferably less than about 70%, less than about 60% or less than about 50% of the number of nucleotides of an identified MAR sequence are within the scope of the present invention. Those constructs preferably comprise the 3′ terminal region of the identified MAR, even more preferably at least about 5%, about 6%, about 7%, about 8%, about 9% or about 10% of the 3′ terminal region of an identified MAR/MAR sequence. However, MAR constructs that contain the 5′ terminal region of the identified MAR are also within the scope of the present invention

### Synthetic MARs

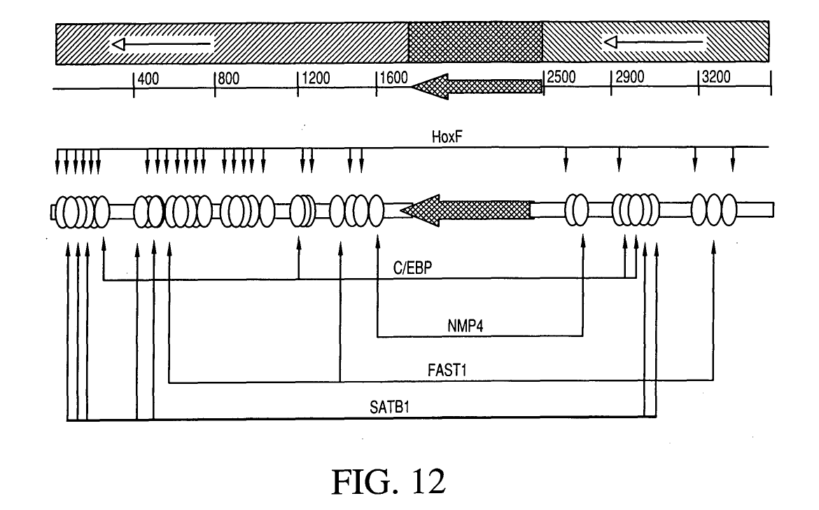

The rearrangement of the human 1_{—}68 MAR showed that a 223 bp fragment of the Hox-rich region located at the 3′ end of the forward hatched portion of an isolated MAR, retains, in certain embodiments, the activity of the full-length region. This suggests that this portion may, in certain embodiments of the invention, be of importance in cooperating with other elements. FIG. 12 shows an array potential transcription factor binding sites of MAR 1_{—}68, as predicted by the MATInspector software. The position of the C/EBP, NMP4, FAST1, SATB1, and HoxF binding sites are shown as examples, illustrating their enrichment in the 5′ (forward hatched) flanking sequence.

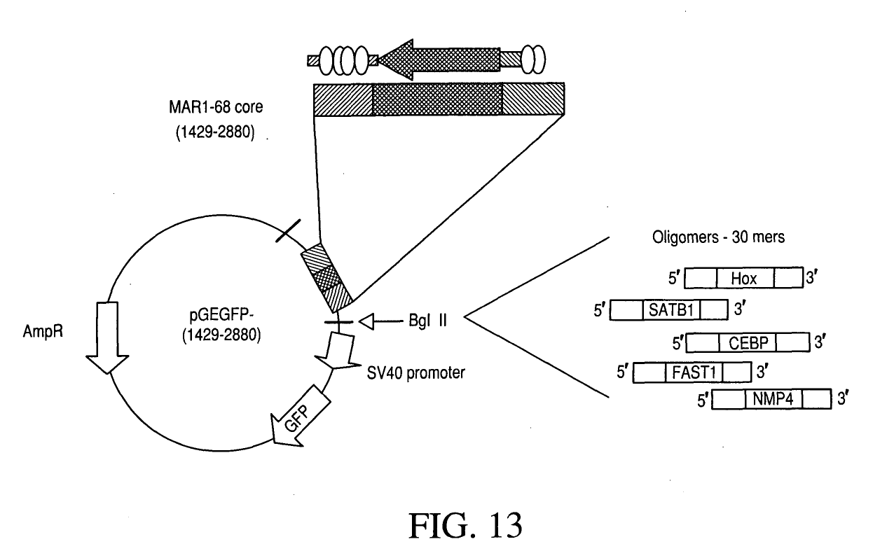

The findings of a possible cooperation between the AT-rich bent DNA region and transcription factor binding sites in human MAR 1_{—}68 prompted the construction of MARs/MAR constructs comprising the AT-rich region of MAR 1-68 adjacent to one or several transcription factor binding sites. FIG. 13 depicts a map of the plasmid used to test for the activity of synthetic MARs constructed from the assembly of a core (MAR 1429-2880) comprising an AT-rich region as well as TFBS of the identified MAR at each end of the AT-rich region and chemically synthesized DNA binding sites for the transcription factors placed upstream of a promoter for green fluorescent protein (GFP). FIG. 13 shows in particular that transcription factor binding sites were inserted between the AT-rich domain and the SV40 promoter driving the expression of the GFP transgene, mimicking the situation found in FIG. 9, where MAR portions containing binding sites are interposed between the promoter and the bent DNA region in the most favorable settings (construct 6). Table 4 shows the DNA sequence of the chemically-synthesized oligonucleotides that were used.

Binding sites for the C/EBP, NMP4, FAST1, SATB1, and HoxF (also called Gsh) transcription factors were identified from the MAR 1-68 sequence (FIG. 12). These binding sites as they occur in MAR 1-68 were used without change (FAST1, C/EBP, HOXF/Gsh), or they were corrected in case they had one or two mismatches as compared to the consensus (i.e. perfect) sequence (HoxF, SatB1, NMP4).

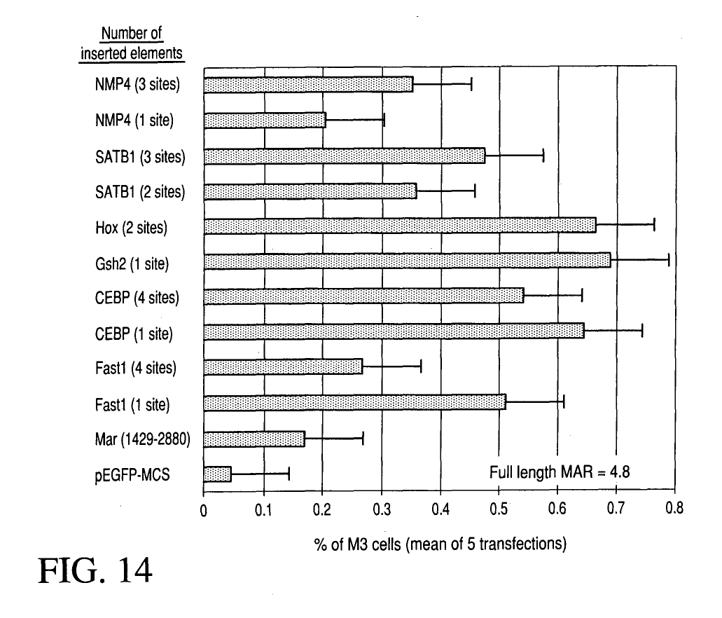

As can be seen from FIG. 14, the addition of the, here, synthetic bind sites provided in almost all cases some, in certain cases, significant transcriptional enhancement compare to the core MAR sequence comprising the AT-rich region. C/EBP and Hox or Gsh2 were most active, followed by SatB1 and Fast1, while one NMP4 site had no detectable effect.

FIG. 14 shows the surprising result that insertion of a core sequence, here MAR 1429-2880 based on MAR 1_{—}68, that is flanked by binding sites of the identified MAR the AT-rich region is based on, did not bring considerable improvement in expression, but a MAR construct further comprising one or more binding sites, in particular when inserted downstream the AT-rich core, but upstream of a promoter resulted in a considerable enhancement of protein expression/production by the gene under the control of the promoter (here identified by the % of M3 cells).

While, in preferred embodiments the additional binding sites are downstream the AT-rich core, but upstream of the promoter, other configurations, such as, but not limited to, a location upstream the AT-rich region, within the AT-rich region, adjacent to the AT-rich region of the core or downstream of the gene, are also within the scope of the present invention.

In a preferred embodiment, certain combinations of protein binding sites, either synthetic or isolated, are contemplated, such as combinations of two different protein binding sites, combinations of three different protein binding sites, combinations of four, five, six, seven, eight, nine, ten or more protein binding sites. These combinations may be multimerized, in full or in part. In a preferred embodiment, the combination comprises Hox/Gsh and SATB1. The insertion of these combinations or multimerized combinations, e.g., between the core and the appropriate promoter, may increase the occurrence of high expressor clones about two fold or more, such as, but not limited to, about three, four, five, six, seven, eight, nine fold or more, preferably about 10 fold or more, even more preferably, about 11, 12, 13, 14, 15, 16, 17, 18, 19 fold or more or about 20 or even about 25 or about 30 fold or more, relative to the occurrence of high expression clones when vectors not comprising a MAR construct/MAR sequence are used under otherwise equivalent conditions.

In sum, MAR constructs can be assembled from building blocks. These building blocks may include or be based on regions, such as sequence specific regions, of identified MARs or parts thereof, synthetic building blocks (including modifications to optimize their functionality), such as a series of chemically synthesized transcription factor binding sites (TFBS), building blocks from or based on non-MAR sequences, or building blocks of or based on MAR sequences of different species or genera. In a preferred embodiment, such MARs comprise AT-rich regions coupled to TFBS regions or specific transcription factor DNA-binding site combinations as those shown in Table 5. The person skilled in the art will appreciated that these principles are not limited to the particular sequences or to the binding sites disclosed herein, and that other derivatives, homologues or sequence combinations are also within the scope of the present invention.

As mentioned above, the MAR constructs, expression systems and/or kits of the invention can be used for protein production. Here a MAR construct may be included in a vector comprising a gene for a protein of interest, for example insulin, under the control of a promoter. The vector is introduced into a cell and the cells are grown. The process is then scaled-up for large scale batch production of insulin. High insulin production, e.g. 3 to 5 times higher than without the MAR construct, can be maintained over three weeks.

As mentioned above, the MAR constructs, expression systems and/or kits of the invention can be used for in vitro and/or in vivo gene therapy and in cell and tissue replacement therapy. E.g., in vitro gene therapy a MAR construct may be included in a vector comprising a gene defective in the patient in need of in vitro gene therapy under the control of a promoter. Subsequently the MAR construct is introduced into cells, such as bone marrow cells of the patient. After transformation with the MAR construct, the bone marrow cells are introduced into the patient and expression of the gene of interest may precede at a level 5 times higher than without the MAR construct. An effective amount of protein may thus be expressed.

In in vivo gene therapy, a vector comprising the MAR construct may be directly introduced into the cells of a patient in need thereof, e.g. by injection.

Similarly, an expression systems of the present invention can be introduced into a stem cell for engraftment for tissue regeneration or for, e.g., neuronal cell therapy for neurodegenerative diseases. Non-limiting examples of stem cells, which can be used in this embodiment of the invention, are hematopoietic stem cells (HSCs) and mesenchymal stem cells (MSCs) obtained from bone marrow tissue of an individual at any age or from cord blood of a newborn individual. The stem cells are transfected with an expression system according to the present invention and successful transformants can be transplanted or reintroduced into a patient in need of the cell therapy or tissue regeneration therapy. Several methods are available for obtaining transformed stem cells, e.g., Nucleofection® (Cell Line Solution V (VCA-1003), amaxa GmbH, Germany).

Transgenic animals, which can produce a wide variety of proteins including antibodies that bind to human antigens, can be produced by known methods (e.g., but not limited to, U.S. Pat. Nos. 5,770,428, 5,569,825, 5,545,806, 5,625,126, 5,625,825, 5,633,425, 5,661,016 and 5,789,650 issued to Lonberg et al.). The expression systems and MAR constructs can be employed in protein production via, e.g., transgenic cattle, sheep, goats or pigs, typically by secretion of the protein into a biological fluid (e.g., milk). See, e.g., U.S. Pat. No. 5,750,172 to Meade et al. See also U.S. Pat. No. 6,518,482 to Lubon et al. for the production of transgenic animals.

### Examples

The invention will be further described in the following examples, which do not limit the scope of the invention set forth in the claims, the summary of the invention or elsewhere herein. The materials, methods, and examples are illustrative only and are not intended to be limiting. With the guidance provided herein, the person skilled in the art will be able make modifications, additions and improvements all of which are within the scope of the present invention.

### S/MARs Prediction of Mouse Genome: SMAR Scan I

All mouse chromosome sequences corresponding to the NCBI m34 mouse assembly were compiled and analyzed with SMAR Scan I. Low and high stringency screens were performed using either a threshold for the DNA bending criterion of 3.6 degrees and a minimal window size of 300 bp, or a threshold of 4.2 degrees and a minimal window size of 100 bp, respectively.

Low stringency analysis via SMAR Scan I of the whole mouse genome yielded a total of 1496 putative S/MARs (candidate MARs), representing a total of 622,410 bp (0.024% of the whole mouse genome). Table 1 shows for each chromosome: its size, its number of genes, its number of predicted MARs (candidate MARs), its MARs density per gene and the average distance in kb between S/MARs. This table reveals that there are various gene densities per predicted S/MAR (candidate MAR) on different chromosomes (with a standard deviation representing around 50% of the mean of the density of genes per MARs). The fold difference between the higher and the lower density of genes per MAR is 6 without considering the chromosome Y, which is extremely rich in predicted MAR (candidate MARs) relative to its size and its number of gene, indicating a strong and unexpected bias in the distribution of these MARs. Table 1 also shows that the average distance between S/MARs (kb per S/MAR) is variable (standard deviation represents 38% of the mean of kb per S/MAR and the fold difference between the higher and the lower density of kb per S/MAR is 8.3). The chromosomes 10, 11, X and Y contribute significantly to the high standard deviation of these densities.

SMAR Scan I has been originally tuned for human sequences and thus yields few MARs with mouse genomic sequences when using the most stringent parameters: therefore, the default cutoff values were adjusted for the high stringency screen (threshold of 4.2 degrees for the DNA bending criterion) to a minimum size of contiguous hits to be considered as MAR, using a window of 100 bp instead of 300 bp. Analysis by SMAR Scan I of the mouse genome predicted 49 “super” MARs with a value>4.2 degrees for the DNA bending criterion.

TABLE 1
Number of S/MARs and “super” S/MARs predicted per mouse chromosomes.

| | Number of | Size of the | Number of | Number of | Density of | |
|---|---|---|---|---|---|---|
| | genes per | chromosome | S/MARs | “super” S/MARs | genes | Kb per |
| Chromosome | chromosome | (millions bp) | predicted | predicted | per S/MAR | S/MAR |
| 1 | 1′367 | 195 | 92 | 4 | 14.9 | 2′120 |
| 2 | 1′613 | 183 | 81 | 3 | 19.9 | 2′259 |
| 3 | 1′119 | 160 | 88 | 3 | 12.7 | 1′818 |
| 4 | 1′439 | 155 | 69 | 2 | 20.9 | 2′246 |
| 5 | 1′423 | 151 | 94 | 3 | 15.1 | 1′606 |
| 6 | 1′341 | 150 | 70 | 7 | 19.2 | 2′143 |
| 7 | 1′994 | 142 | 82 | 3 | 24.3 | 1′732 |
| 8 | 1′169 | 128 | 107 | 3 | 10.9 | 1′196 |
| 9 | 1′293 | 124 | 57 | 4 | 22.7 | 2′175 |
| 10 | 1′107 | 130 | 167 | 5 | 6.6 | 778 |
| 11 | 1′762 | 122 | 44 | 1 | 40.0 | 2′773 |
| 12 | 824 | 118 | 61 | 3 | 13.5 | 1′934 |
| 13 | 978 | 115 | 57 | 1 | 17.2 | 2′018 |
| 14 | 984 | 119 | 80 | 1 | 12.3 | 1′488 |
| 15 | 877 | 104 | 57 | 4 | 15.4 | 1′825 |
| 16 | 752 | 98 | 69 | 1 | 10.9 | 1′420 |
| 17 | 1′103 | 93 | 62 | 0 | 17.8 | 1′500 |
| 18 | 576 | 91 | 35 | 1 | 16.5 | 2′600 |
| 19 | 787 | 61 | 27 | 0 | 29.1 | 2′259 |
| X | 1′186 | 164 | 47 | 0 | 25.2 | 3′489 |
| Y | 22 | 2 | 50 | 0 | 0.4 | 40 |
| Sum | 23′716 | 2′605 | 1′496 | 49 | 366 | 39′420 |
| Mean | 1′129 | 124 | 71 | 2 | 17 | 1′877 |
| Sd | 430 | 43 | 30 | 2 | 8 | 716 |

The number of genes per chromosome corresponds to the NCBI m34 mouse assembly (National Center for Biotechnology Information). Chromosome sizes are the sum of the corresponding mouse Reference Sequence contig lengths.

### Use of Newly Identified Mouse MARs to Increase Production of Recombinant Proteins

Five MAR elements were selected from the putative MARs (candidate MARs) obtained with the high stringency screen of the complete mouse genome with SMAR Scan. They were cloned in plasmid vectors from mouse genomic DNA bacterial artificial chromosomes purchased from the Children's Hospital Oakland Research Institute (CHORI, http://bacpac.chori.org/).

These newly-identified mouse MARs were named S4, S8, S15, S32 and S46 (according to the order of identification by SMAR Scan I, “super” MARs S1 to S49). The human MARs 1_{—}3, 1_{—}6, 1_{—}9, 1_{—}42, 1_{—}68, 3_S5 and X_S29 have been previously identified, the MARs 168 and X_S29 being the most potent human elements (Mermod et al. “High efficiency gene transfer and expression in mammalian cells by a multiple transfection procedure of MAR sequences,” WO2005/040377, see also U.S. Patent Publication 20070178469 to Mermod et al). These MARs were inserted into the pGEGFP control vector upstream of the SV40 promoter and enhancer driving the expression of the green fluorescent protein and these plasmids were transfected into cultured CHO cells, as described previously [Girod P A, Zahn-Zabel M and Mermod N]. Expression of the transgene was then analyzed in the total population of stably transfected cells using a fluorescent cell sorter (FACS) machine. FIG. 1 shows the effect of various S/MARs on the production of recombinant green fluorescent protein (GFP). Populations of CHO cells transfected with a GFP expression vector pGEGFP comprising or not comprising a MARs as indicated by a fluorescence-activated cell sorter (FACS®), and typical profiles are shown. Only the most potent human MARs 1_{—}68 and X_S29 are shown in this figure. The profiles display the cell number counts as a function of the GFP fluorescence levels. Horizontal bars representing the cell subpopulations M1, M2 and M3 with fluorescence values smaller than 2 (M1), or greater than 10² (M2) or 10³ (M3) relative light units are indicated.

As can be seen from FIG. 1, all of the newly identified mouse MARs increased the expression of the transgene significantly above the expression driven by the GFP alone without MAR, the “super” mouse MAR S4 being the most potent of all MARs shown.

TABLE 2
Detailed analysis of the GFP fluorescence from polyclonal populations of CHO cells.

| Construct | Mean ± SEM | | CV ± SEM | | M1 (%) ± SEM | | M2 (%) ± SEM | | M3 (%) ± SEM | |
|---|---|---|---|---|---|---|---|---|---|---|
| pGEGFP | 2.88 | 0.21 | 144.3 | 6.0 | 63.64 | 2.29 | 2.07 | 0.39 | 0.04 | 0.00 |
| 1_68 | 13.88 | 1.24 | 83.7 | 1.8 | 34.20 | 1.76 | 22.62 | 2.15 | 1.05 | 0.19 |
| X_S29 | 13.70 | 1.43 | 85.0 | 3.5 | 35.36 | 2.27 | 22.91 | 1.70 | 0.98 | 0.20 |
| S4 | 20.63 | 1.49 | 80.7 | 2.9 | 32.14 | 2.57 | 34.19 | 0.78 | 2.87 | 0.33 |
| S8 | 8.92 | 0.43 | 92.7 | 0.3 | 39.36 | 0.33 | 13.04 | 1.49 | 0.50 | 0.06 |
| S15 | 4.43 | 0.19 | 128.3 | 4.3 | 57.12 | 2.62 | 7.75 | 0.19 | 0.27 | 0.10 |
| S32 | 4.99 | 0.65 | 116.0 | 6.6 | 51.70 | 3.13 | 6.21 | 1.72 | 0.22 | 0.07 |
| S46 | 11.30 | 0.93 | 88.0 | 3.8 | 35.95 | 2.35 | 18.44 | 1.04 | 0.79 | 0.11 |

CHO cells were co-transfected with an antibiotic selection plasmid and with the pGEGFP reporter construct, or with pGEGFP derivatives containing either the human MARs 1_68 and X_S29, or the indicated mouse S4, S8, S15, S32 or S46 MAR. The polyclonal population of stably transfected cells was selected for antibiotic resistance during two weeks and tested for GFP fluorescence by FACS analysis as displayed in FIG. 1. The Table displays the mean fluorescence value, its coefficient of variation, and the percentile of cells showing fluorescence values smaller than 2 (M1), or fluorescence values greater than 10² (M2) or 10³ (M3) relative light units. These results are the average values and standard error of the mean (SEM) was obtained from three independent experiments.

The transcriptional activity of the most potent human MARs 1_{—}68 and X_S29 was compared to the ones obtained with the newly identified mouse MARs. Five mouse MARs were initially tested via GFP expression assays, and they were all found to increase the expression of GFP to different levels. Mouse MARs S15 and S32 are relatively the least transcriptionally active MARs (−2 fold increase compared to GFP alone), S8 and S46 showed a medium activity (3-4 fold increase) and MAR S4 displayed very high transcriptional activity (7 fold increase). Moreover, mouse MAR S4 is the most potent of all MARs tested in this study. Comparison between the human MAR 1-68 and mouse MAR S4 transcriptional activity reveals a 50% increase of the mean fluorescence of the whole population (Gmean M0) and of the high GFP-producing cells (M2), whereas the percentile of very high GFP-producing cells (M3) was 175% higher with mouse MAR S4. The homogeneity of the whole population in terms of GFP fluorescence (CV M0) was always 1-2% lower with mouse MAR S4, which is advantageous because it indicates greater stability of the cell productivity.

After this first round of cloning, it was sought to be determined if highly active MAR elements can be consistently obtained from the mouse genome. Thus, two additional mouse MARs (S6 and S10) were cloned and characterized. These new mouse MARs were inserted into the pGEGFP control vector and analyzed by FACS as above. Mouse MAR S10 appeared to be also more potent than the best human MARs in all the different parameters analyzed by FACS, and is nearly as active transcriptionally as MAR S4 to increase overall expression.

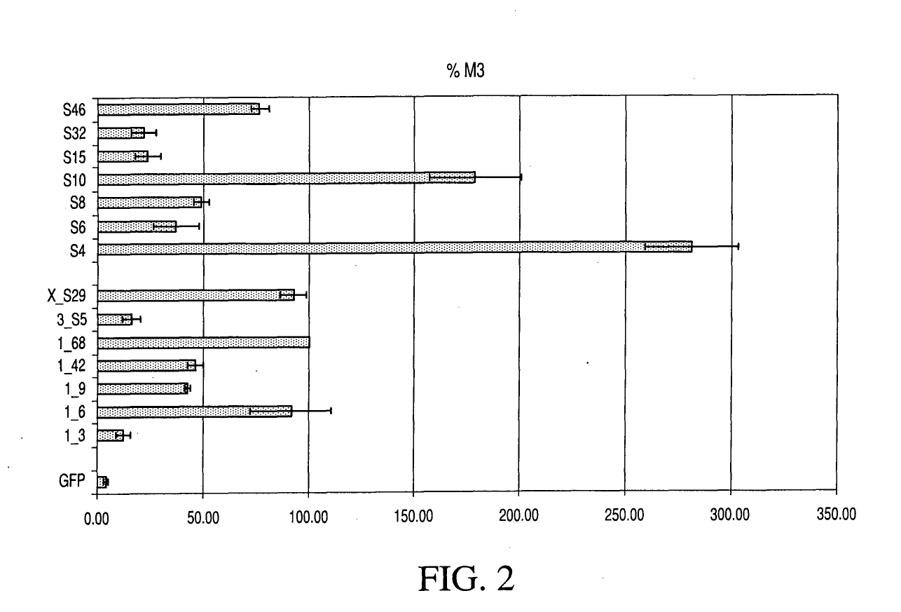

To assess very high producers, the percentile of M3 cells normalized to the one obtained for the human MAR 1_{—}68. The result are presented in FIG. 2. FIG. 2 shows the effect of various human and mouse S/MAR elements on the percentile of very high producers (% M3) of recombinant green fluorescent protein (GFP). Populations of CHO cells transfected with a GFP expression vector containing or not containing a MAR element as indicated, were analyzed by a fluorescence-activated cell sorter (FACS®). The percentile of very high producers was normalized to the one obtained with the best human MAR for this criterion, the MAR 1_{—}68, whose value was set to 100. Mouse MARs S10 and S4 gave on average 80% and 180% more very high producer cells than the human MAR 1_{—}68, respectively. Overall, from this comparison of 7 mouse MARs with 7 human MARs, it was concluded that higher expression was achieved from CHO cells using rodent MARs.

### Assessment of Potency pf Newly Identified Mouse MARs in Different Cell Types

The potency of the S4 MAR was assessed in CHO cells. In addition, EGFP expression vectors comprising either human MAR 1-68, mouse MAR S4 or no MAR were transfected stably in human HeLa cells and EGFP fluorescence was analyzed. FIG. 3 shows the effect of various human 1_{—}68 and mouse S4 MAR elements on the expression of recombinant green fluorescent protein (GFP). Populations of HeLa cells were transfected and analyzed as described for Table 2. In a comparison of the potency of S4 and 1-68 MAR in HeLa cells, S4 was found to out perform 1_{—}68 in several respects: S4 yielded higher average GFP fluorescence (Average Gmean M0) as well as more cells in the medium and high expression range (M1 and M2 respectively), and a lower variability of expression (Average CV M0). No cells were found in the very high expression range (M3) using HeLa cells.

### Enhanced Expression of Monoclonal Antibodies Using Mouse MARs

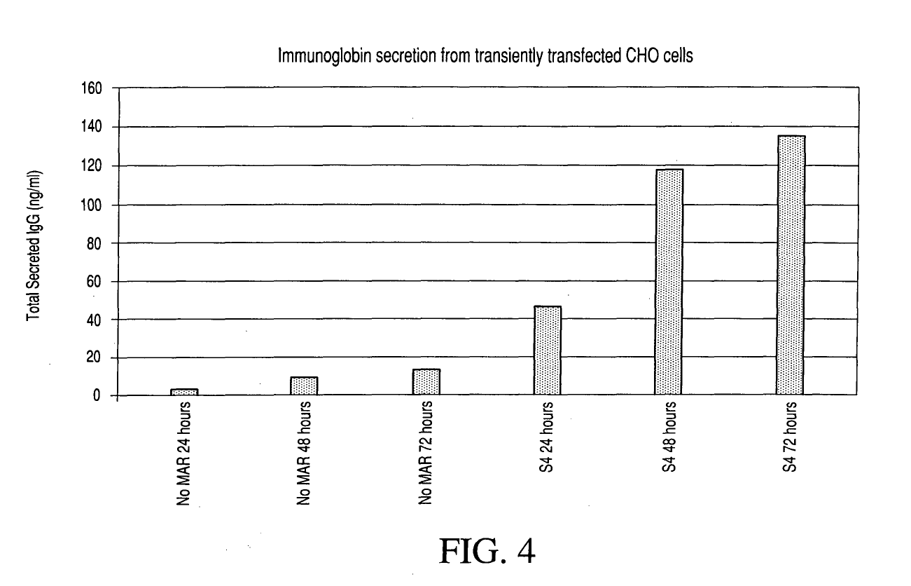

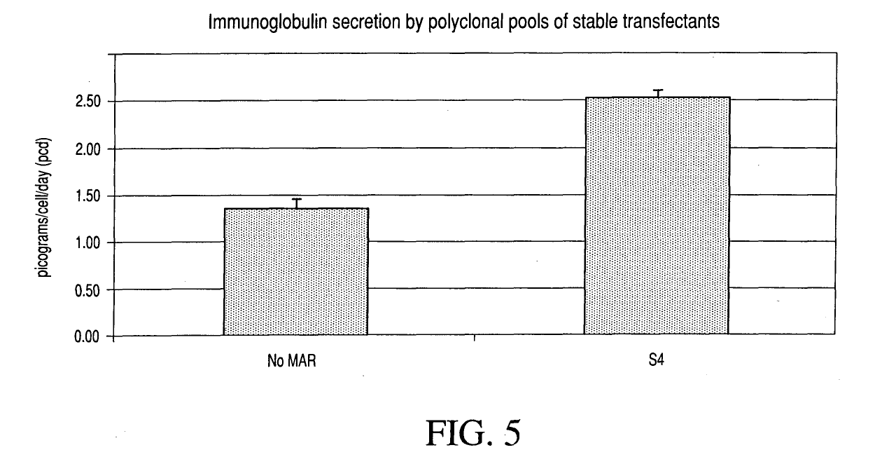

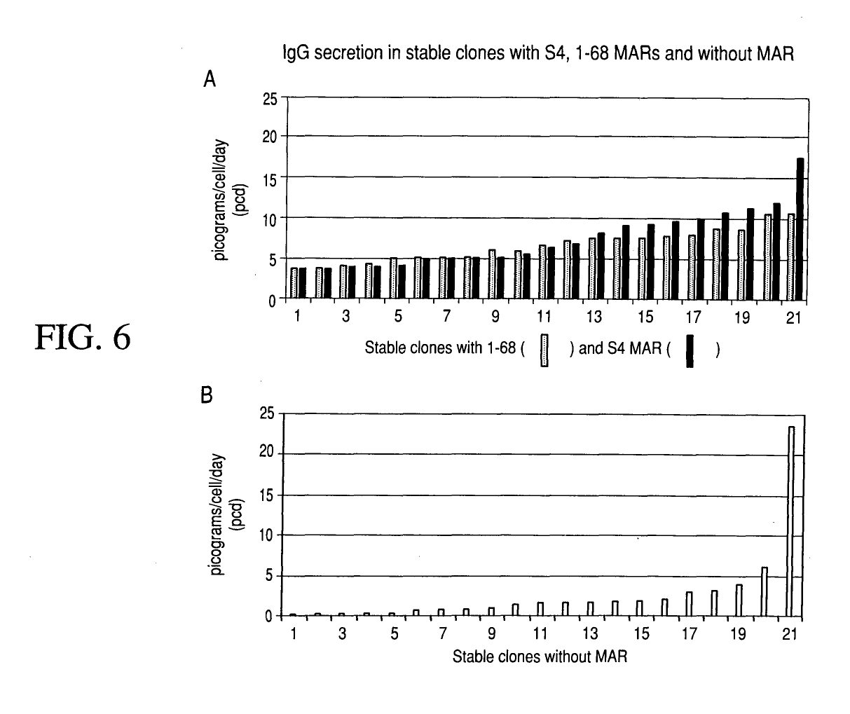

To determine if mouse MARs, in particular the most potent ones, can be used to augment the production of proteins for pharmaceutical applications, they were inserted in the pMZ37 and pMZ59 vectors encoding the heavy and light chains of a Rhesus-D-recognizing immunoglobulin [Miescher S, Zahn-Zabal M, De Jesus, M, Moudry, R, Fisch, I, Vogel, M, Kobr, M, Imboden, M A, Kragten, E, Bichler, J, Mermod, N, Stadler, BC, Amstutz, H., Wurm, F]. These plasmids were transfected in CHO cells, selection and immunoglobulin assays were performed as described previously [Girod P A, Zahn-Zabel M and Mermod N]. FIG. 4 shows the effect of S/MAR elements on the production of recombinant monoclonal antibodies. Here, CHO cells were transfected with the above-mentioned vectors driving expression of IgG heavy and light chains without MAR (no MAR), or with the MAR S4 added in cis. IgG titers were measured in the supernatants after 24, 48 and 72 hours. In addition and as depicted in FIG. 5, stable clones were generated from a population of CHO cells transfected with the above mentioned vectors driving expression of IgG heavy and light chains without MAR (no MAR), or with the MAR S4 added in cis. After selection, secreted IgG titers were measured in the medium and specific productivity was assayed by cell counting. FIG. 6 (A) shows results obtained after stable individual clones were generated by limiting dilution from a population of CHO cells transfected with vectors driving expression of IgG heavy and light chains without MAR (no MAR), or with the MAR S4 added in cis. After selection, secreted IgG titers were measured in the medium and specific productivity was assayed by cell counting. Also included are comparative results obtained with MAR 1_{—}68 as well as in (B) results obtained with clones not comprising a MAR. The results obtained and depicted in FIGS. 3 to 6 indicate that the newly identified mouse MARs, in particular MAR S4, can be used to boost the production of pharmaceutical proteins, such as monoclonal antibodies, in transient transfectants (FIG. 4) and in stable transfectants (FIGS. 5 and 6). Stable clones with specific productivities around or above 5 pg/cell/day (pcd) can be readily identified from an analysis of a few candidate clones when using MAR S4 (FIG. 6(A)). Indeed the average productivity of the 21 best clones with or without the MAR S4 was 7.28±0.78 pcd (FIG. 6(A)) and 2.61±1.09 pcd, respectively. These results stand in contrast to the titer levels obtained with the known chicken lysozyme MAR (less than 1.5 mg/L) or without MAR (less than 0.5 mg/L). In particular, these results indicate that the newly identified mouse MARs can be used to boost the production of proteins of pharmaceutical use such as, but not limited to, monoclonal antibodies, rendering mouse MARs, such as MAR S4, particularly interesting for the production of recombinant proteins.

Expression Stability with Human MAR 1_{—}68

MAR 1_{—}68 was used to demonstrate that the expression of genes that are produced by clones, not containing MARs are gradually silenced, equivalent clones containing MARs not only maintain high level expression over time, but silent cells recover expression.

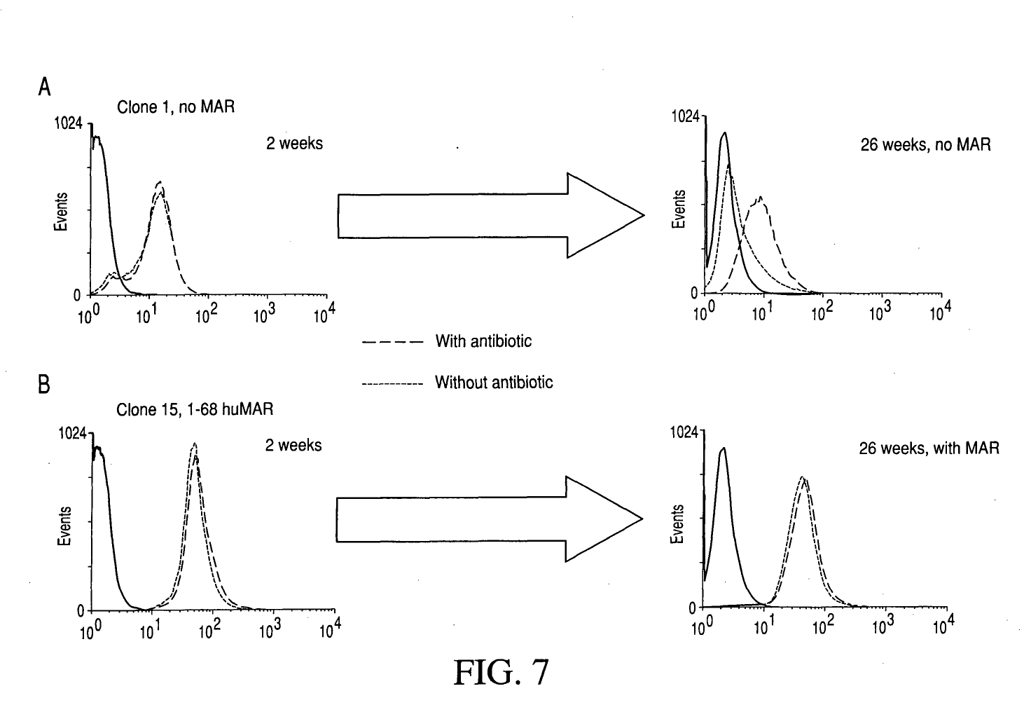

FIG. 7 shows the co-transfection of the pEGFP expression plasmid comprising MAR 1-68 into CHO cells with a G418 antibiotic resistance gene, and stably expressed cells were selected in the presence of G418 for three weeks, as described in Girod et al., 2005. Cell clones were obtained by limiting dilution and 9 individual clones were analyzed for GFP fluorescence. A typical clone expressing GFP was selected from each of the two populations for further analysis and cultured further up to 26 weeks in the presence or absence of antibiotic selection. Profiles represent GFP fluorescence levels (x axis) and number of cell counts on the y axis after two weeks of culture on the left-hand side, while profiles on the right were obtained from cells cultured for 26 weeks. As can be seen, the clone lacking the MAR shows decreased GFP fluorescence level in the absence of antibiotic after 26 weeks relative to the level after two weeks, while the clone comprising a MAR could maintain the GFP fluorescence level at week 26 with or without antibiotic selection, making MAR comprising expression systems useful for the stable expression of a gene of interest.

### Modularity of MARs and Relevance for Gene Expression Enhancement

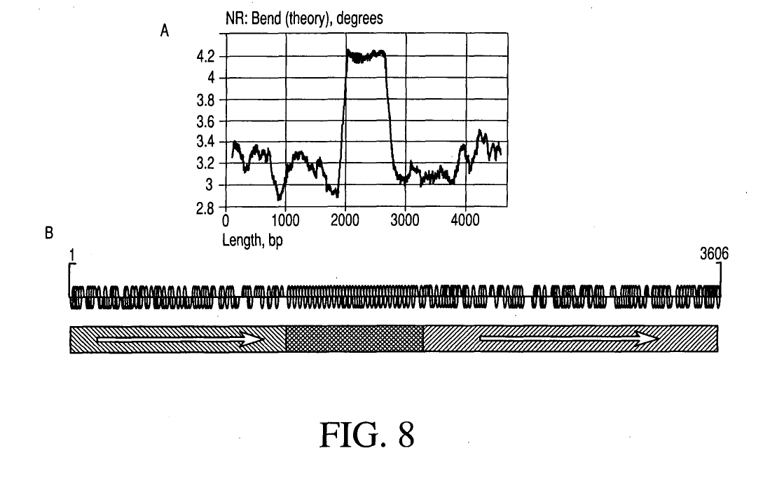

A structural analysis of MARs revealed DNA sequence regions/modules that each contribute to enhanced gene expression. FIG. 8 depicts the results obtained via a structural analysis of the 1_{—}68 MAR. In FIG. 8(A) shows that a central AT-rich region dictates bent DNA in the MAR 1_{—}68 locus. FIG. 8(B) shows that this AT-rich region is surrounded by regions rich in transcription factors binding sites as identified by MatInspector (Cartharius, Frech et al. 2005). Precisely 729 potential TFBSs were detected by MatInspector along the MAR sequence. The lower part of FIG. 8(B) shows attributes a coding to the identified regions.

FIG. 9 (A) shows 1_{—}68 MAR and on the left hand site different MARs that incorporate regions or parts of 1_{—}68 MAR and change the order and/or orientation of the regions or parts thereof and/or duplicate such regions or parts thereof. On the right hand side the degree of transcriptional augmentation achieved by constructs 1 to 16 is shown as well as the transcriptional augmentation achieved with MAR 1_{—}68 or no MAR. All MAR sequences shown were inserted upstream of the promoter driving the eGFP gene marker. The arrows depicts the orientation of the regions or fragments thereof relative to the wild type MAR sequence depicted in FIG. 8. The sequences surrounding the AT-rich region are shown as backward hatched box with arrow (on the left) and a forward hatched box with arrow (not to scale; right). The bent region is shown as a crosshatched box.

FIG. 9 (B) shows the bending pattern of the MAR that corresponds to construct 6 in FIG. 9.A. These bending pattern were determined via SMARScan I.

FIG. 9 (C) shows the results of a MatInspector [Cartharius, Frech et al. 2005] analysis. Potential transcription factors binding sites (TFBSs) were identified by MatInspector [Cartharius, Frech et al. 2005]. 731 potential sites are detected by MatInspector along the MAR sequence. On the bottom of FIG. 9(C) construct 6 is shown using the coding corresponding to FIG. 8(B) and FIG. 9(A). The coding of the bottom portion of this Figure corresponds to the one shown and discussed in FIG. 9(A).

The experiments depicted in FIG. 9 show that none of the regions display full MAR activity by themselves. For example, enhancement of DNA transcription resulting from the naturally occurring human 1_{—}68 MAR to the full extent requires three distinct sequences (FIG. 8): a 1189 bp segment that contains binding sites for multiple transcription factors (i.e. CEBP) (FIG. 9A top, shown as a backward hatched box with an arrow, an intrinsically bent DNA that is dictated by a 763 bp symmetric AT-rich region (alternating A and T) (FIG. 9A top, crosshatched box), and an additional 1648 bp segment which includes many HoxF and SATBI binding sites (FIG. 9A top, shown as a forward hatched box with an arrow).

FIG. 9 shows that the improvement of human 1_{—}68 MAR by moving away the bent DNA to augment the size of the transcription factors binding site region upstream the promoter region. To achieve this augmentation, the transcription factor binding site (TFBS) region, here a Hox-rich region (SEQ ID No. 19) (hereinafter, the forward hatched region with arrow), adjacent to the AT-rich (SEQ ID. No. 18) region was adjoined to the CEBP-rich region (SEQ ID No. 17) (hereinafter, also the backward hatched region (FIG. 9)). Comparison of transcriptional enhancement activity of the different resulting MAR constructs as depicted on the right hand side of FIG. 9A shows that the orientation of the forward hatched region with arrow was important for the transcriptional augmentation (compare constructs 5 and 6). The data shown also strongly indicate that a distal transcriptional control element itself restricts transcription initiation in the downstream chromatin. Given that a 223 bp fragment (SEQ ID No. 20) located at the 3′-end of the forward hatched region with arrow retains the full activity of the region in construct 7, suggests that this important portion must, in this case, cooperate with the bent region and the 5′-end of the remainder (nucleotides 1-1425) of the element in construct 6. Two HMG-I/Y sites were found to be located nearby this terminus.

### Modularity of Mouse MARs and Reduction of Size

Based on the findings with human 1_{—}68 MAR, the S4 MAR was also analyzed for modules, in particular those responsible for its transcriptional activity. This analysis was also performed with the goal of reducing the size of the S4 MAR, which is relatively long. Thus, several MARs were constructed from the S4 MAR (Table 3) and characterized (FIG. 10). FIG. 10 shows on the left hand side the specific MAR S4 construct, and on the right hand side, the effect of various MAR S4 constructs on the expression of recombinant green fluorescent protein (GFP) as revealed by the analysis of the average fluorescence of the whole population (Avg Gmean M0). Populations of CHO cells transfected with a GFP expression vector comprising or not comprising a MAR construct as indicated, were analyzed by flow cytometry with a FACScalibur cytometer (Becton Dickinson). The average fluorescence of the whole population was normalized to the one obtained with the human MAR 1_{—}68, whose value was set to 100, while GFP indicates expression in the absence of MAR. Other MAR constructs are named according to their base content as compared to the full length 1547 bp S4 MAR (see Table 3). The dotted box indicates the AT rich bent region of MAR S4. S_{—}41-4662-Luc5489 indicates a construct where the terminal (3′) 795 base pair were removed and replaced by part of the luciferase gene (black box). Interestingly and as can be seen in FIG. 10, it was found that a 1624-bp EcoRI fragment can be deleted from S4 MAR (S4-1-703_{—}2328-5457) without significant loss of its MAR activity. However, deletion of the promoter-proximal 795-bp fragment, or replacement of this sequence by a fragment of the luciferase gene of similar length (S4_{—}1-4661; S4_{—}1-4661-Luc5489), induced a complete loss of this activity. This indicates that certain variants of the mouse S4 MAR can display high activity, while being of shorter in length, thus making it of more convenient size for, e.g., vector design and transfer.

TABLE 3
MAR S4 constructs in pGEGFP vector

| S4 construct | Description |
|---|---|
| S4 (SEQ ID No. 3) | 5457 bp AvaI insert from bacmid RP23-444A8 |
| S4_1-703_2328-5457 | Internal deletion of a 1624-bp EcoRI fragment |
| (SEQ ID No. 4) | |
| S4_1-2395_4121-5457 | Internal deletion of a 1724-bp HindIII fragment |
| (SEQ ID No. 5) | |
| S4_1-4661 (SEQ ID | Internal deletion of a 795-bp BglII |
| No. 8) | fragment with the BglII site present |
| | in the MCS of the vector |
| S4_1-4661-Luc5489 | S4_1-4661 construct with 828-bp |
| | BglII-digested PCR product |
| | from the luc gene |
| S4_4662-5457 (SEQ | 795-bp BglII fragment with the BglII |
| ID No. 9) | site present in the MCS of the vector |
| S4_2328-4661 (SEQ | 2333-bp EcoRI-BglII fragment of S4 |
| ID No. 7) | |
| S4_2328-5457 (SEQ | 3129-bp EcoRI-AvaI fragment of S4 |
| ID No. 6) | |

### Activity of the 3′ Terminal MAR Sequences

To further analyze the activity of the 3′ end sequences of MAR S4, this portion of the MAR was further dissected by removing or duplicating portions of it. FIG. 11 shows the effect of various MAR S4 derivatives on the expression of recombinant green fluorescent protein (GFP) as revealed by the analysis of the average fluorescence of the whole population (Avg Gmean M0). Populations of CHO cells were generated and assayed as described above. Interestingly, one such derivative, having a truncated 3′ end (4658-5054 vs. 4658-5457 of the original MAR S4), displayed, on average, a slightly higher transgene expression compared to the longer original MAR S4 sequence (104% vs 100%). This indicates that more potent as well as shorter derivatives of MAR elements can be obtained.

### Synthetic MARs

FIG. 12 shows a map of potential transcription factor binding sites [of 1_{—}68 MAR], as predicted by the MATInspector software. The position of the C/EBP, NMP4, FAST1, SATB1, and HoxF (also called Gsh) binding sites are shown as examples, illustrating their enrichment in the 5′ forward hatched flanking sequence. These binding sites as they occur in MAR 1-68 were used without change (FAST1, C/EBP, HOXF/Gsh), or they were corrected in case they had one or two mismatches as compared to the consensus (i.e. perfect) sequence (HoxF, SatB1, NMP4).

The findings of a possible cooperation between the AT-rich bent DNA region and transcription factor binding sites in human MAR 1_{—}68 prompted the construction of synthetic MARs comprising the AT-rich portion of MAR 1-68 adjacent to one or several transcription factor binding sites. FIG. 13 depicts a map of the plasmid used to test for the activity of synthetic MARs constructed from the assembly of a core comprising an AT-rich region (MAR 1429-2880) and chemically synthesized DNA binding sites for the transcription factors placed upstream of a promoter and green fluorescent protein (GFP). FIG. 13 shows that transcription factor binding sites were inserted between the AT-rich core and the SV40 promoter driving the expression of the GFP transgene, mimicking the situation found in FIG. 9, where MAR portions containing binding sites are interposed between the promoter and the bent DNA region in the most favorable settings. Table 4 shows the DNA sequence of the chemically-synthesized oligonucleotides that were used.

TABLE 4
Putative transcription factor binging sites from human MAR 1_68

| | |
|---|---|
| FAST1 | GAT CCA GTA CTC ATG TTC ATT TTC TCT AGA |
| SEQ ID No. 11) | GT CAT GAG TAC AAG TAA AAG AGA TCT CTAG |
| (Perfect) | |
| CEBP | GAT CCA GTA CTG TTT GGG AAA TTC CAT GGA |
| (SEQ ID No. 12) | GT CAT GAC AAA CCC TTT AAG GTA CCT CTAG |
| (Perfect) | |
| HOXF (GSH) | GAT CCA GTA CTC CCC TAA TTC AGA CAT GCA |
| (SEQ ID No. 13) | GT CAT GAG GGG ATT AAG TCT GTA CGT CTAG |
| (Perfect) | |
| HOXF | GAT CCA GTA CTA ATA ATA AAA TAC CCG GGA |
| SEQ ID No. 14) | GT CAT GAT TAT TAT TTT ATG GGC CCT CTAG |
| (1 mismatch) | |
| SATB1 | GAT CCA GTA CTT TAT TAT AAT ATG TTA ACA |
| (SEQ ID No. 15) | GT CAT GAA ATA ATA TTA TAC AAT TGT CTAG |
| (2 mismatches) | |
| NMP4 | GAT CCA GTA CTG GGA AAA AAA TCG TCG ACA |
| (SEQ ID No. 16) | GT CAT GAC CCT TTT TTT AGC AGC TGT CTAG |
| (1 mismatch) | |

Paired 30-mer oligomers with cohesive ends that were cloned into a vector containing the AT-rich core region of MAR 1_68. The italizied base pairs are sequences of the transcription factor binding sites (most conserved bases underlined) and flanking sequences that originate from the MAR 1_68. Sequences in regular font are linker or adapter sequences that do not correspond to MAR 1_68 sequences. On these linker sequences, oligomers with 1 or 2 mismatches from MAR 1_68 were modified to match the consensus.

FIG. 14 shows the transcriptional enhancement by synthetic MARs constructed as described in FIG. 13. The inserted elements contain 1 or several protein DNA-binding sites in addition to the core, as indicated. Transfection of plasmids containing one or several binding sites in addition to the core sequence comprising an AT-rich region (AT-rich core) indicated that inclusion of binding sites increased transcriptional enhancement in comparison to the AT-rich core alone, and that C/EBP and Hox or Gsh2 were most active, followed by SatB1 and Fast1, while one NMP4 site had no detectable effect.

Different mixtures of active binding sites were also tested, to determine if synergistic effects may be observed. To do so various combinations of oligonucleotides containing binding sites for the different transcription factors were mixed in DNA ligation reactions, and the precise order and arrangement of binding sites were determined by DNA sequencing. The obtained combinations are showed in Table 5:

TABLE 5
Synthetic MAR constructs containing various heteromultimers
of transcription factor binding sites

| | | Total no. |
|---|---|---|
| Clone No. | Transcription factor sites | of sites |
| 1 | Gsh, 2(SATB1) | 3 |
| 2 | SATB1, Hox | 2 |
| 3 | SATB1, Fast1 | 2 |
| 4 | 2(Hox), SATB1, Hox | 4 |
| 6 | Gsh, 2(SATB1), CEBP, Hox | 5 |
| 7 | 2(Fast1), 2(Gsh), SATB1 | 5 |
| 8 | Hox, SATB1, Hox, Gsh, SATB1, Hox | 6 |
| 9 | Gsh, 2(Fast1) | 3 |
| 10 | 3(CEBP), SATB1, Hox, Fast1 | 6 |
| 11 | Hox, Fast, Hox, Fast | 4 |
| 12 | Hox, SATB1, Hox, Gsh, Hox, Hox | 6 |
| 13 | 2(Hox), 3(SATB1), Fast, CEBP, Hox, CEBP | 9 |
| 14 | Gsh, Gsh | 2 |
| 15 | CEBP, Hox, Hox | 3 |

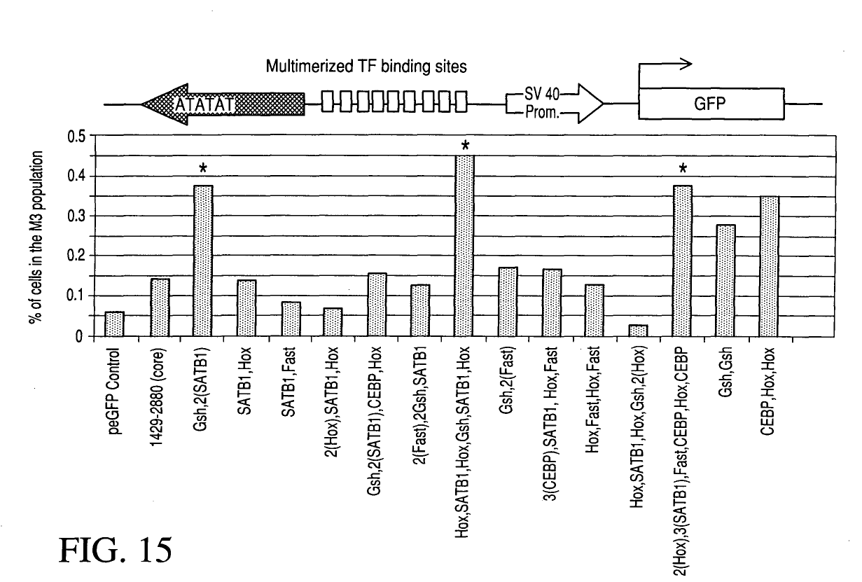

The resulting plasmids were tested by transfection as before. FIG. 15 shows the transcriptional enhancement by synthetic MARs constructed with the DNA binding site combinations shown in Table 5. The most active combinations are indicated by a star sign, and the occurrence of HoxF/Gsh2 or SatB1 sites is indicated. The results shown in FIG. 15 indicate that the activity of the synthetic MARs does in this instance not depend on the number of inserted binding sites, but that particular combinations of binding sites show high enhancement activities, while others lack activity or even repress gene expression. Constructs with higher activities comprised in this case combinations of Hox/Gsh2 and SATB1 proteins, and the most active construct is exclusively composed of these elements. Insertion of this synthetic MAR increased the occurrence of high expressor clones approximately 10-fold as compared to the pEGFP control vector devoid of any MAR sequence.

### BIBLIOGRAPHY

Abdurashidova G, Danailov B, et al., “Localization of proteins bound to a replication origin of human DNA along the cell cycle.” EMBO J22: 4294-4303, 2003.
Aladjem, M I and Fanning E., “The replicon revisited: an old model learns new tricks in metazoan chromosomes.” EMBO Rep 5(7): 686-91, 2004.
Allen G C, Spiker S, Thompson W F, Use of matrix attachment regions (MARs) to minimize transgene silencing, Plant Mol Biol., 43(23):361-376, 2000.
Amati B and Gasser S M, Chromosomal ARS and CEN elements bind specifically to the yeast nuclear scaffold, Cell, 54:967-978, 1988.
Amati B and Gasser S M, Drosophilia scaffold-attached regions bind nuclear scaffolds and can function as ARS elements in both budding and fission yeasts, Mol. Cell. Biol., 10:5442-5454, 1990.
Bell S P, “The origin recognition complex: from simple origins to complex functions.” Genes Dev 16: 659-672, 2002.
Bode J, Schlake T, Rios-Ramirez M, Mielke C, Stengart M, Kay V and KlehrWirth D, Scaffold/matrix-attached regions: structural properties creating transcriptionally active loci, Structural and Functional Organization of the Nuclear Matrix: International Review of Cytology, 162A:389-453, 1995.
Bode J, Benham C, Knopp A and Mielke C, Transcriptional augmentation: modulation of gene expression by scaffold/matrix-attached regions (S/MAR elements), Crit Rev Eukaryot Gene Expr, 10(1):73-90, 2000.
Bode J, Stengert-Iber M, Kay V, Schlake T and Dietz-Pfeilstetter A, Scaffold/matrix-attached regions: topological switches with multiple regulatory functions, Crit. Rev. Euk. Gene Exp., 6:115-138, 1996.
Bodnar J W, A domain model for eukaryotic DNA organization: a molecular basis for cell differentiation and chromosome evolution, J. Ther. Biol., Vol. 132:479-507, 1988.
Boulikas T, Nature of DNA sequences at the attachment regions of genes to the nuclear matrix, J. Cell Biochem., 52:14-22, 1993.
Boulikas T, Chromatin domains and prediction of MAR sequences. In Structural and Functional Organization of the Nuclear Matrix: International Review of Cytology, Academic Press, Orlando, 162A:279-388, 1995.
Breyene P, Van Montagu M and Gheyseu G, The role of scaffold attachment regions in the structural and functional organization of plant chromatin, Transgenic Res., Transgenic Res., 3:195-202, 1994.
Breyne P, Van Montagu M, Depicker A and Gheysen G, Characterization of a plant scaffold attachment region in a DNA fragment that normalizes transgene expression in tobacco, Plant Cell, 4:463-471, 1992.
Cartharius, K., K. Frech, et al., MatInspector and beyond: promoter analysis based on transcription factor binding sites, Bioinformatics 21: 2933-42, 2005.
Gasser S M and Laemmli U K, Cohabitation of scaffold binding regions with up-stream/enhancer elements of three developmentally regulated genes of D. Melanogaster, Cell, 46:521-530, 1986.
Girod P A, Zahn-Zabal M and Mermod N, Use of the chicken lysozyme 5′ matrix attachment region to generate high producer CHO cell lines, Biotechnol. Bioeng., 91(1):1-11, 2005.
Kas E and Chaslin L A, Anchorage of the Chinese hamster dihydrofolate reductase gene to the nuclear scaffold occurs in an intragenic region, J. Mol. Biol., 198:677-692, 1987.
Kay V and Bode J, Detection of scaffold-attached regions (SARs) by in vitro techniques; activities of these elements in vivo. In Methods in Molecular and Cellular Biology: Methods for studying DNA protein interactions: an overview, WileyLiss, New York, 5:186-194, 1995.
Kim J M, Kim J S, Park D H, Kang H S, Yoon J, Baek K and Yoon Y, Improved recombinant gene expression in CHO cells using matrix attachment regions, J. Biotechnol., 107(2):95-105, 2004.
Kwaks T H, Otte A P, Employing epigenetics to augment the expression of therapeutic proteins in mammalian cells. Trends Biotechnol. 24:137-42, 2006.
Labrador, M. and V. G. Corces, Setting the boundaries of chromatin domains and nuclear organization, Cell 111: 151-54, 2002.
Levine, M. and R. Tjian, Transcriptional regulation and animal diversity, Nature 424: 147-151, 2003.
Mielke C, Kohwi Y, KohwiShigematsu T and Bode J, Hierarchical binding of DNA fragments derived from scaffold-attached regions: correlation of properties in vitro and function in vivo, Biochemistry, 29:7475-7485, 1990.
Miescher S, Zahn-Zabal M, De Jesus M, Moudry R, Fisch I, Vogel M, Kobr M, Imboden M A, Kragten E, Bichler J, Mermod N, Stadler B C, Amstutz H, Wurm F, CHO, Expression of a Novel Human Recombinant IgG1 anti-Rh D Antibody Isolated by Phage
Display, Brit. J. Haematol., 111, 157-166, 2000.
National Center for Biotechnology Information (http://www.ncbi.nih.gov).
PhiVan L and Stratling W H, Dissection of the ability of the chicken lysozyme gene 5′ matrix attachment region to stimulate transgene expression and to dampen position effects, Biochemistry, 35:10735-10742, 1996.
Razin S V, Functional architecture of chromosomal DNA domains, Crit Rev Eukaryot Gene Expr, 6:247-269, 1996.
Stefanovic D, Stanojcic S et al., In vitro protein—DNA interactions at the human lamin B2 replication origin, J Biol Chem 278: 42737-42743, 2003.
Strick R and Laemmli U K, SARs are cis DNA elements of chromosome dynamics: synthesis of a SAR repressor protein, Cell 83(7): 1137-48, 2005.
Vogelstein B, Pardoll D and Coffey D, Supercoiled loops and eukaryotic DNA replication, Cell, 22:79-85, 1980.
You Z, Ishimi Y, et al., Thymine-rich single-stranded DNA activates Mcm4/6/7 helicase on Y-fork and bubble-like substrates, EMBO J 22: 6148-6160 (2003).
Zahn-Zabal M, Kobr M, Girod P A, Imhof M, Chatellard P, de Jesus M, Wurm F and Mermod N, Development of stable cell lines for production or regulated expression using matrix attachment regions. J Biotechnol, 87(1): 29-42, 2001.

1. An expression system for high-level expression of at least one gene comprising:
a promoter for operably liking a nucleotide sequence encoding a gene of interest, and
at least one non-human mammalian MAR nucleotide sequence for enhancing expression of a said gene in a cell transformed with said expression system,
wherein said non-human mammalian MAR nucleotide sequence increases expression of said gene about 2, about 3, about 4, about 5, about 6, about 7, about 8, about 9, about 10 fold or more upon transformation of said cell with said construct.

2. The expression system of claim 1, wherein an expression cassette comprising said promoter and said nucleotide sequence encoding a gene of interest is operably linked to the promoter.

3. The expression system of claim 1, wherein said at least one non-human mammalian MAR nucleotide sequence is a rodent MAR nucleotide sequence, such as a mouse or hamster MAR nucleotide sequence.

4. The expression system according to claim 1, wherein said non-human mammalian MAR nucleotide sequence comprises:
(i) SEQ ID No. 3, SEQ ID No. 10 or a functional fragment thereof; or
(ii) a nucleotide sequence having about 80%, about 90%, about 95% or about 98% sequence identity with any of the sequences of (i).

5. The expression system of claim 1, wherein said gene is expressed in a non-human mammalian cell such as a rodent cell, in particular a mouse or hamster cell, or in a human cell, such as a HeLa cell.

6. The expression system of claim 1, wherein said at least one non-human mammalian MAR nucleotide sequence acts in cis or trans on said gene.

7. A method for enhanced protein production in a cell comprising
providing a human or non-human mammalian cell,
introducing the expression system of any of the above claims into said cell so that gene expression is increased about 2, about 3, about 4, about 5, about 6, about 7, about 8, about 9, about 10 fold or more.

8. An isolated and purified nucleic acid molecule comprising:
(a) the nucleotide sequence of SEQ ID No. 3 or SEQ ID No. 10 or a functional fragment thereof, or
(b) a nucleotide sequence that has at least about 80%, about 90%, about 95% or about 98% sequence identity with the sequence of (a) and has MAR activity.

9. A method for identifying non-human mammalian MAR sequences comprising:
providing at least one non-human mammalian nucleic acid molecule, preferably a non-human mammalian genome or a part thereof,
subjecting said nucleic acid molecule to a scanning procedure for MAR sequences comprising:
setting a window size for nucleic acid molecules to be evaluated,
selecting at least 1 or at least 2, preferably 3, more preferably 4 or more MAR associated features,
setting threshold values for sequences displaying this/these feature(s), and
selecting MAR candidate nucleotide sequences exceeding these threshold values,
ascertaining that said non-human mammalian MAR nucleotide sequence increases expression of a gene about 2, about 3, about 4, about 5, about 6, about 7, about 8, about 9, about 10 fold or more upon transformation of a human and/or non-human mammalian cell via an expression system comprising said non-human mammalian MAR nucleotide sequences.

10. A method according to claim 9, wherein said at least one feature may be a DNA bending angle, major groove depth, minor groove width, melting temperature or combinations thereof.

11. The method of claim 10, wherein DNA bending angle values include between about 3 and about 5° (radical degree), preferably between 3.8 about 4.4°, including about 3.9, about 4.0, about 4.1, about 4.2 and about 4.3°.

12. The method of claim 10, wherein major groove depth values are between about 8.9 to about 9.3 Å and minor groove width values are between about 5.2 to about 5.8 Å, preferably, the major groove depth values are between about 9.0 to about 9.2 Å, including about 9.1 Å and the minor groove width values may be between about 5.4 to about 5.7 Å, including about 5.5 Å and about 5.6 Å.

13. The method of claim 10, wherein the melting temperature is between about 55 and about 75° C., in particular between about 55 and about 62° C. including about 56, about 57, about 58, about 59, about 60 and about 61° C.

14. The method of claim 10, wherein DNA bending angle values are about 4.0 to about 5.0°, including about 4.1, about 4.2, about 4.3, about 4.4, about 4.5, about 4.6, about 4.7, about 4.8 and about 4.9°.

15. The method of claim 14, wherein said DNA bending angle values are combined with window values ranging from about 50 bps to about 150 bps, including, e.g., about 80 bps, about 100 bps and about 120 bps.

16. The method of claims 10, wherein the DNA bending angle value times a window value are between about 320 and 1320 such as, about 420 and about 1220, about 520 and about 1120, about 620 and about 1020, about 720 and about 920, the major groove depth value times the window value are between about 900 and about 4000, such as about 1200 and 3700, about 1500 and about 3400, about 1800 and about 3100, about 2100 and about 2800 and/or minor groove depth value times the window size are between about 500 and about 2500, such as about 750 and about 2250, about 1000 and about 2000, about 1250 and 1750.

17. The method of claim 9, further comprising:
providing experimentally validated MARs of human or non-human origin;
determining said threshold values using said experimentally validated MARs of human or non-human origin.

18. A MAR construct comprising:
(a) (i) an isolated nucleotide sequence comprising at least part of a terminal region of an identified MAR, and
(ii) a further isolated nucleotide sequence comprising about 10%, about 15%, about 20%, about 25%, about 30% or more of said identified MAR or another identified MAR; or
(b) (i) a nucleotide sequence having about 90%, about 95%, about 96%, about 97% about 98%, about 99% sequence identity with the nucleotide sequence of (a) (i), and
(ii) a nucleotide sequence having about 70%, about 80%, preferably about 90%, about 95%, about 96%, about 97% about 98%, about 99% sequence identity with the nucleotide sequence of (b) (i).

19. The MAR construct according to claim 18, wherein said nucleotide sequence in (a) (ii) comprises an AT-rich region.

20. A MAR construct according to claim 18, wherein said MAR construct comprises less than about 90%, preferably at less than about 80%, even more preferably less than about 70%, less than about 60% or less than about 50% of a number of nucleotides of an identified MAR sequence.

21. A MAR construct according to claim 18, wherein said MAR construct comprises about the same or at least about 110% of a number of nucleotides of an identified MAR sequence.

22. A MAR construct comprising
regions of an identified MAR sequence in consecutive arrangement, wherein an order and/or an orientation differs from that of an identified MAR sequence.

23. The MAR construct of claim 22, wherein said regions comprise at least one AT-rich region and at least one binding site region.

24. The MAR construct of claim 23, wherein said MAR construct further comprises at least part of at least one binding site region and wherein said at least part of said at least one binding site region is, optionally, from said identified MAR sequence.

25. The MAR construct of claim 24, wherein said identified MAR sequence is a human or a mouse MAR.

26. The MAR construct of claim 22, wherein said regions of the identified MAR sequence or parts thereof have about 70% sequence identity, about 80% sequence identity, about 90% sequence identity, about 95% sequence identity or about 98% sequence identity with regions of the naturally occurring human 1_{—}68 MAR or mouse MAR S4 or parts thereof.

27. The MAR construct of claim 22, wherein said regions correspond to bps 1 to 1189, 1190 to 1952 and 1953 to 3600, respectively of a naturally occurring human 1_{—}68 MAR.

28. The MAR construct of claim 22, wherein the regions are sequence-specific regions.

29. A MAR construct comprising:
(a) a core nucleotide sequence comprising
(i) at least one isolated or synthetic AT-rich region of an identified MAR sequence; or
(ii) at least one AT rich region having at least at least 80%, 85%, 90%, 95%, 98% or 99% sequence identity with the AT-rich region of (a) (i),
(b) a nucleotide sequence comprising
at least one DNA protein binding site adjacent to said nucleotide sequence of (a), wherein said binding site is
(i) a DNA protein binding site of a further identified MAR sequence,
(ii) a DNA protein binding site of the identified MAR sequence of (a), wherein said DNA protein binding site is, in the identified MAR sequence, situated outside the core nucleotide sequence of (a), or
(iii) a first DNA protein binding site present in the core of (a), but adjacent to at least one further DNA protein binding site, wherein the first and at least one of said further DNA protein binding sites are not adjacent in the core of (a), or
(iv) a DNA protein binding sites of a non-MAR sequence.

30. The MAR construct of claim 29, wherein said construct enhances expression of a gene operably linked to a promoter about 2, about 3, about 4, about 5, about 6, about 7, about 8, about 9, about 10 fold or more upon introduction of said MAR construct into a cell.

31. The MAR construct of claim 29, wherein said MAR construct is less than 500 nucleotides, preferably less than about 250 nucleotides, even more preferably less than about 200, about 150 or about 100 nucleotides long.

32. The MAR construct of claim 29, wherein said core nucleic acid sequence of (a) comprises at least one TFBS of said identified MAR, wherein said at least one TFBS flanks said AT-rich region in the identified MAR unilaterally or bilaterally.

33. The MAR construct of claim 29, wherein said at least one DNA protein binding sites in (b) is a TFBS and is modified by 1, 2, 3, 4, 5 or more substitutions, additions and/or deletions and/or has, in full or part, been synthesized.

34. The MAR construct of claim 33, wherein said TFBS that flank said AT-rich region is modified by 1, 2, 3, 4, 5 or more substitutions, additions and/or deletions.

35. The MAR construct of claim 33, wherein said TFBS is an optimized TFBS with no known natural counterpart.

36. The MAR construct of claim 29, wherein said binding sites are selected from a group consisting of SATB1, NMP4, HOX, HOXF, Gsh, CEBP, Fast1 and SATB1 or a combination of two or more of these transcription factors.

37. The MAR construct of claim 29, wherein a series of said DNA protein binding sites of (b) are adjacent to said nucleic acid sequence of (a).

38. The MAR construct of claim 29, wherein said MAR construct is an enhanced MAR construct.

39. A expression system comprising
at least one of the MAR constructs of any of the above claims, and, optionally,
a promoter and at least one restriction enzyme binding site for introducing a nucleotide sequence of interest under the control of said promoter.

40. A cell comprising an expression system of claim 39.

41. A transgenic non-human animal comprising an expression system of claim 39.

42. A kit comprising:
the expression system of claim 39, and
instructions how to use said expression system.

43. A method for enhancing expression of a gene comprising
providing a expression system comprising said gene under the control of a promoter and of a MAR construct of claim 39;
transfecting a cell with said expression system so that the expression of said gene is enhanced.

44. A method of claim 43, wherein said expression system further enhances stability of expression of said gene.

45. (canceled)

46. (canceled)
